(19) United States
(12) Patent Application Publication
(10) Pub. No.: US 2006/0068490 A1
(43) Pub. Date: Mar. 30, 2006

(54) Flow-through chemical and biological sensor

(76) Inventors: Cha-Mei Tang, Potomac, MD (US); Platte T. Amstutz III, Vienna, VA (US)

Correspondence Address: MCDERMOTT, WILL & EMERY, 4370 LA JOLLA VILLAGE DRIVE, SUITE 700, SAN DIEGO, CA, 92122, US

(21) Appl. No.: 11/073,430

(22) Filed: Mar. 4, 2005

Related U.S. Application Data
(60) Provisional application No. 60/550,442, filed on Mar. 5, 2004.

(51) Int. Cl. C12M 1/34

(52) U.S. Cl. 435/287.2

(57) ABSTRACT

The invention provides a mixing flow apparatus. The mixing flow apparatus consists of a waveguide and a mixing flow chamber; the waveguide having a higher index of refraction material than its surroundings for propagation of a signal, and the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet. Also provided is a detection apparatus. The detection apparatus consists of a waveguide, a mixing flow chamber and a radiation detector; the waveguide having a higher index of refraction material than its surroundings for propagation of a signal; the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet, and the radiation detector being disposed facing the direction of oncoming propagated signal from the waveguide. The detection apparatus can include an illumination source.

This application is based on, and claims the benefit of, U.S. Provisional Application No. 60/550,442, filed Mar. 5, 2004, entitled “Flow-Through Chemical and Biological Sensor,” the entire disclosure of which is incorporated herein by reference.

This invention was made with government support under grant numbers R43 CA094430, R43 AI052684 and R43 EB001731 awarded by the National Institutes of Health. The United States Government has certain rights in this invention.

### BACKGROUND OF THE INVENTION

The invention relates generally to optical sensors and, more particularly to waveguide flow-through sensors using a chemical or molecular recognition species to detect or capture the analytes, such as bacteria, viruses, spores, oocysts, cells, cell fragments, proteins, receptor and binding proteins, nucleic acid (DNA, cDNA, RNA), oligonucleotides, antibodies, enzymes, antibiotics, peptides, carbohydrates, tumor and disease markers, toxins, organic and inorganic compounds and parts thereof.

Biological and chemical sensors are used to detect analytes, such as bacteria, viruses, spores, oocysts, cells, cell fragments, proteins, receptor and binding proteins, nucleic acid (DNA, cDNA, RNA), oligonucleotides, antibodies, enzymes, antibiotics, peptides, carbohydrates, tumor and disease markers, toxins, organic and inorganic compounds, hereafter referred to as “analytes.”

These analytes can be present in (1) air, (2) food, (3) liquid, such as water, buffer, serum, whole blood, urine, saliva, sweat, etc., (4) tissue, (5) feces, (6) environmental samples, (7) man made products, etc. In order to be detected, these analytes must first be captured and then identified. The capturing methods can be a physical trap, chemical reaction, or molecular recognition species. Molecular recognition species are typically a protein (e.g., antibody, an antigen target for an antibody analyte, cell receptor or binding protein), nucleic acid (e.g., DNA, cDNA, or RNA), an oligonucleotide, a carbohydrate, an aptamer, ribozyme, enzyme, antibiotic, ligand, cell, cell fragment and the like.

Once the analyte is captured, a signal in the form of optical, electrical, magnetic, thermal, chemical, or change of physical properties can be produced and detected in order to indicate the presence and/or identity of the analyte. The invention herein relates to optical-based detection, utilizing waveguides.

Even with all the available detection methods, the detection of low concentrations of analyte, i.e., a small amount of analyte in a large volume of sample, is still a challenge. For example, in the detection of pathogens in drinking water, foods and environmental samples, microbiologists face a “needle-in-a haystack” challenge. Even after preparatory processing, the sample size can be quire large (>25 mL) compared to the volume required by most analytical methods and instruments.

Even though some DNA hybridization methods based on nucleic acid amplification, such as polymerase chain reaction (PCR), can detect a single bacteria under ideal conditions in their amplification volumes, the amplification volume, however, is small (<10 μl), a small fraction of the sample size. Some immunoassays can only process a few micro-liters of the whole sample. Splitting of the sample is required to reduce its volume, which also reduces the amount of analyte in the test volume, sometimes to a level that is below the detection limit of the method or instrument. The ideal situation is to be able to process the whole sample.

Flow-through sensors, such as those that flow the sample along the outside of solid optical fiber or inside hollow capillary tube, are capable of flowing the sample rapidly, provided the cross-sectional area of the flow channel is sufficiently large. However, only a small portion of the analyte in the sample comes in contact with the analyte sensing surface. Thus, the limit of detection remains high.

The reason is that micro-channel flow in uniform channels is laminar, meaning that the velocity vector of the flow is parallel to the wall, and the motion in the transverse direction is only by diffusion. The ability for the sensor to function requires the analyte to come in contact with the capturing structure on the surface of the channels. Analyte suspended in the fluid far from the surface of the waveguide will not come in contact with the capture surface of the waveguide because of the passive laminar flow property.

Some methods to overcome this consist of flowing the sample very slowly, allowing the reaction to incubate for a long time, and/or using small channel dimensions. However, these techniques either increase the testing time or reduce the volume of sample that can be tested.

Thus, there exists a need for a sensor with enhanced interaction, between the constitutive elements of a sample fluid and the analyte capture surface, and thus improved efficiency and sensitivity of detection. The present invention satisfies this need and provides related advantages as well.

### SUMMARY OF THE INVENTION

The invention provides a mixing flow apparatus. The mixing flow apparatus consists of a waveguide and a mixing flow chamber; the waveguide having a higher index of refraction material than its surroundings for propagation of a signal, and the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet. Also provided is a detection apparatus. The detection apparatus consists of a waveguide, a mixing flow chamber and a radiation detector; the waveguide having a higher index of refraction material than its surroundings for propagation of a signal; the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet, and the radiation detector being disposed facing the direction of oncoming propagated signal from the waveguide. The detection apparatus can include an illumination source.

### BRIEF DESCRIPTION OF THE DRAWINGS

FIGS. 1a, 1b and 1c are schematic representations of a top view, side view and end view of a mixing flow-through sensor according to one embodiment of the invention.

FIGS. 2a-2h are cross-sectional representations of the waveguide according to several embodiments of the invention.

FIGS. 3a and 3b are schematic representations of the top views of the compact mixing flow-through sensors according to other embodiments of the invention, where the side walls of the mixing flow chamber have different shapes.

FIGS. 4a and 4b are cross-sectional representations of a mixing flow-through sensor according to one embodiment of the invention at two axial locations. The body of the mixing flow channel has a three-dimensional variation.

FIGS. 4c and 4d are cross-sectional representations of a mixing flow-through sensor according to an embodiment of the invention at two axial locations. The body of the mixing flow channel has another three-dimensional variation.

FIGS. 5a and 5b are cross-sectional representations of a mixing flow-through sensor according to one embodiment of the invention at two axial locations. The radiation transmissive top surface of the mixing flow chamber is also the waveguide and the mixing is achieved by three-dimensional undulating bottom and side surfaces of the mixing flow chamber.

FIGS. 6a and 6b are schematic representations of a compact mixing flow-through sensor according to one embodiment of the invention at two different axial locations. The mixing flow chamber contains two waveguides and they are illuminated by two radiation sources. The mixing flow is produced by the undulating side walls in combination with the waveguides.

FIG. 7 present side view of a mixing flow-through sensor according to another embodiment of the invention. The sensing system 900 comprises waveguide members 901 that are disposed inside elongated body of mixing flow chamber 940. The end of the waveguide 903 is unobscured by the waveguide wall 934 to let the emission light out to the detector. Elongated body 940 includes top transmissive member 920 and bottom light absorbing member 930 and an inlet 960 and outlet 961. The bottom wall member 930 is undulating. The excitation light 950 is collimated but not perpendicular to the long direction of the waveguide.

The mixing flow is produced by the undulation of the bottom wall in combination with the waveguide.

FIGS. 8a, 8b and 8c are schematic representations of a top view, side view and end view of a multi-analyte mixing flow-through sensor according to one embodiment of the invention, respectively. There is one waveguide in each mixing flow chamber.

FIGS. 9a, 9b and 9c are schematic representations of a top view, side view and end view of a flow-through sensor according to one embodiment of the invention, respectively. There are many waveguides in the mixing flow chamber. The flow is perpendicular to the length of the waveguide. The mixing of the flow is produced primarily by the waveguide. This embodiment can be for detection of a single analyte or multi-analyte. Two detector systems can be used.

FIG. 10 is a side view of the multi-waveguide flow-through sensor where the waveguide are positioned to further enhance mixing of the fluid as it flows pass the waveguides.

FIGS. 11a, 11b and 11c are schematic representations of a top view, side view and end view of a multi-analyte flow-through sensor according to one embodiment of the invention, respectively. There are a number of mixing flow chambers. There are many waveguides in each mixing flow chamber. The flow is perpendicular to the length of the waveguide. Again, the mixing of the flow is produced primarily by the waveguide.

FIG. 12 shows the options of the fluid flow for the embodiment shown in FIGS. 11a, 11b and 11c.

FIGS. 13a, 13b and 13c are schematic representations of the top view, side view and end view of a curved mixing flow chamber and curved waveguide.

FIG. 14 is a top representation of a multiple mixing flow-through sensor according to one embodiment of the invention with curved surfaces.

FIG. 15 is an end view of a schematic representation of a multi-analyte mixing flow-through sensor according to one embodiment of the invention.

FIG. 16a and 16b are schematic representations of a side view and end view of a mixing flow-through sensor according to one embodiment of the invention where the mixing is accomplished by moving parts.

FIGS. 17a and 17b are schematic representations of the side view and end view of a mixing flow-through sensor according to one embodiment of the invention where the mixing is accomplished by moving parts.

FIG. 18 is a schematic representation of an end view of a mixing flow-through sensor according to one embodiment of the invention where the mixing is accomplished by applying an electric field in part of the flow channel.

FIGS. 19a, 19b and 19c are the bottom, top and end views of the mixing flow—through sensor according to one embodiment of the invention where the fluid is guided to flow in a spiral pattern around the waveguide and the fluid is mixed at the sides of the waveguide.

FIGS. 20a, 20b, 20c and 20d are the bottom, top, end view at one axial location and end view at another axial location of the mixing flow-through sensor according to one embodiment of the invention where the fluid is guided to flow to the left and right of the waveguide and the fluid is mixed at the sides of the waveguide.

### DETAILED DESCRIPTION OF THE INVENTION

The invention provides a flow-through optical sensor wherein the interactions between the constitutive elements of the fluid and the waveguide surface are enhanced in order to maximize the capture of the analyte to the surface of the optical waveguide. Also provided is a sensor having at least one waveguide for each flow channel. Sensors of the invention are provided that allow for the detection of at least one analyte on each waveguide. Further, the invention also provides multiple channels and each channel with at least one waveguide as well as multiple waveguides in each channel. Illumination of the optical sensor and waveguide therein such as by excitation using radiation that is collimated or non-collimated, is also provided. The illumination of the waveguide can be coherent or incoherent. The illumination source can be a point source or a diffused source. Also provided is illumination of the optical sensor and waveguide therein such as by excitation using radiation that is oriented in a direction perpendicular to, at an angle to, or at multiple angles to the surface of the waveguide. The invention further provides increased signal-to-noise ratio by increased transmission of emission radiation and/or reduce the excitation radiation from the waveguide to a detector. Qualitative or quantitative analysis of an analyte or both qualitative and quantitative analysis using the sensor and methods of the invention is additionally provided.

The above embodiments as well as other embodiments of the invention are accomplished by an optical flow-through sensor of the invention. The optical sensor can consist of a waveguide member and a mixing flow chamber having a body. In one specific embodiment, the mixing flow chamber can further contain a first end and a second end, an inlet and an outlet, a radiation transmissive top surface, light absorbing side surfaces and a bottom surface. The body of the mixing flow chamber extends outward from the waveguide member and can be spaced therefrom so as to allow a fluid solution to flow between the inlet and the outlet. The waveguide member can be either secured to the first end and the second end, or to the inlet end and outlet end, or to the sidewalls of the mixing flow chamber. In this specific embodiment of the sensor, the body of the mixing flow chamber can have a radiation transmissive surface and a mixing flow surface. Analytes for capture on the surface of the waveguide member and electromagnetic radiation-emitting materials can be attached, for example, to an analyte recognition element. Upon illumination by an excitation radiation source, radiation is emitted and a portion of the emitted radiation is captured by the waveguide member and propagated to the end of the waveguide to a detector system.

The above embodiments as well as other embodiments of the invention can also be accomplished by an optical flow-through sensor consisting of a mixing flow chamber having a body and a fluid mixing member. This mixing flow chamber can further consist of a first end and a second end, an inlet and an outlet, a radiation transmissive top surface, light absorbing side surfaces and a bottom surface. The body of the mixing flow chamber can allow, for example, a fluid solution to flow between the inlet and the outlet. In this specific embodiment, the waveguide member is part of the mixing flow chamber that has a radiation transmissive surface and the fluid mixing member can be unattached to the mixing flow chamber. The fluid mixing member also can be actuated electronically, mechanically, electromechanically, thermally, electromagnetically, magnetically, by vibration or other energy sources. Analytes for capture on the surface of the waveguide member and radiation-emitting materials are to be attached, for example, to an analyte recognition element. Upon illumination by a radiation source, a distinctive characteristic radiation is emitted, and a portion of the emitted radiation is captured by the waveguide member and can be propagated to the end of the waveguide to a detector system.

As used herein, the term “waveguide” is intended to mean a structure that facilitates the transmission of electromagnetic radiation. Transmission can be facilitated by, for example, using materials that assist electromagnetic radiation propagation along or within a waveguide structure. Transmission also can be facilitated by, for example, imparting directionality on the transmission, reducing loss of an signal, minimizing scatter emission, focusing of a transmitted electromagnetic propagation beam or capture of electromagnetic signal. Other modes of facilitation for electromagnetic radiation transmission are well known to those skilled in the art and also are included within the meaning of the term as it is used herein. Therefore, a waveguide functions, for example, as a conduit of electromagnetic radiation including, for example, optical signals.

The electromagnetic radiation can be guided in the waveguide when the index of refraction of the waveguide is higher than its surrounding. To operate the waveguide surrounded by air, the index of refraction of the waveguide needs to be greater than 1.0. Index of refraction of water is 1.33. Index of refraction of waveguide greater than water would be preferable. For example the index of refraction of glass is about 1.5.

All forms of electromagnetic radiation from infrared to ultraviolet region can be used in connection with a waveguide of the invention. Such forms include, for example, electromagnetic wavelengths within the ultraviolet region of the spectrum at about 50-380 nm, the visible portion at about 380-780 nm, the near-infrared region at about 780-3000 nm, the intermediate infrared region at about 3000-8000 nm as well as longer and shorter wavelengths. Additionally, a waveguide can be composed of, for example, any material and consist of any structural form or shape so long as it facilitates the transmission of electromagnetic radiation. Exemplary materials that can be utilized in a waveguide include, for example, high index of reflection, low transmission loss and non-fluorescent materials such as glass or polystyrene. Other exemplary materials include, for example, polymethyl-methacrylate (PMMA) and quartz. A waveguide can be composed of a single material, mixtures of different materials, rations of the same material or two or more separated materials, for example. Given the teachings and guidance provided herein, those skilled in the art will know, or can determine, whether a waveguide made of a single material, a mixture or distinct and separable materials are beneficial for a particular application.

As used herein, the term “mixing flow chamber” is intended to mean an enclosed or compartmented space that allows the flow of fluids or particulate bodies or other substances that move like fluids and mixing of constituents contained within the chamber. Therefore, a mixing flow chamber allows fluids or other substances with a fluid-like movement behavior to move with a change of place among the constituent particles or parts. The change of place can be continuous or non-continuous as well as regular or sporadic motions. Accordingly, the term “flow chamber” as it is used in reference to a mixing flow chamber refers to the compartmented space in which fluid can flow through. The portion of a flow chamber that compartmentalizes a flow space can be, for example, a body or wall structure or a one or more surfaces forming an encapsulated space. A mixing flow chamber and the flow space corresponding to the flow chamber can take on a variety of sizes and shapes, so long as fluid or other substances with fluid-like movement behavior can change place relative to a position in the mixing flow chamber or relative to other constituents of the fluid or fluid-like substances and so long as the chamber can be configured to produce mixing. Mixing can result by modification of fluid flow directionality or periodicity using, for example, a waveguide, a structure of the flow chamber, other structures or features that augment mixing of fluid, or any combination thereof. Specific examples of a structure of the flow cell include an undulating surface or surfaces of the flow chamber or a non-elongated flow cell shape such as an arc, circle, sphere, periodic shape, or one or more combinations thereof. Specific examples of such other structures or features that augment mixing include a free or unattached waveguide, actuation of movable objects, and application of, for example, an electric field, magnetic field, vibration, heat, electromagnetic or other source of energy, or one or more combinations thereof. For example, the moving objects can be small air bubbles, compressible beads, small magnetic beads or rods. Other means known in the art that facilitate mixing of fluid or fluid-like substances similarly can be used to configure a flow chamber given the teachings and guidance provided herein.

As used herein the term “radiation transmissive” when used in reference to a material, device or apparatus of the invention is intended to mean that the material of a medium that allows electromagnetic radiation to pass or be conveyed through that medium. The term includes a medium that allows passage or conveyance of all wavelengths of electromagnetic radiation including, for example, wavelengths within the ultraviolet region of the spectrum at about 50-380 nm, the visible portion at about 380-780 nm, the near-infrared region at about 780-3000 nm, the infrared region at about 3000-8000 nm as well as longer and shorter wavelengths. Therefore, a radiation transmissive surface functions to admit the passage of radiation.

As used herein, the term “portion” as it is used in reference to a waveguide is intended to mean a part of a waveguide. Therefore, the term refers to less than the whole or entire waveguide.

As used herein, the term “surface” is intended to mean the exterior or outside, or the interior or inside, of an object or body. Therefore, depending on the reference orientation, the term surface can refer to an outer boundary of a structure, an inner boundary of a structure or the entire thickness of a structure when, for example, the structure is a partition dividing contents between spatial locations. A surface also can refer to a portion of a structure. For example, a waveguide can exhibit multiple surfaces. A reference to a surface, as it is used herein, includes some or all of a face of a surface as well as the entire face of a surface. Therefore, the term is intended to include that part of something that is presented to a reference view, a reference orientation, or a reference component of the device or apparatus of the invention. Moreover, it is to be understood that for an optically transmissive structure, a surface can refer to either the exterior, interior or both surfaces when used in reference to optical properties. For example, a reflective surface can be physically contained on an external surface of, for example, a mixing flow chamber, but will also reflect optical signals internally because of the transmissive nature of the structure. Given the teachings and guidance provided herein, those skilled in the art will understand whether external or internal surfaces are functionally distinguishable or alike when reference is made to a particular coating, property or structure.

As used herein, the term “analyte recognition coating” or “analyte recognition element” is intended to refer to a moiety that selectively binds an analyte. Analyte recognition coating or elements are useful for selectively attaching or capturing a target analyte to a waveguide. Attachment or capture includes both solid or solution phase binding of an analyte to an analyte recognition coating. An analyte is attached or captured through a solid phase configuration when the analyte recognition coating or element is immobilized to a waveguide when contacted with an analyte. An analyte is attached or captured through a solution phase configuration when the analyte recognition coating or element is in solution when contacted with an analyte. Subsequent immobilization of a bound analyte-analyte recognition coating or element complex to a waveguide completes attachment or capture to the waveguide. In either configuration, either direct or indirect immobilization of the analyte recognition coating or element to a waveguide can occur. Direct immobilization refers to attachment of the analyte recognition coating or element to a waveguide allowing for capture of an analyte from solution to a solid phase. Immobilization of the analyte recognition coating or element can be directly to a waveguide surface or through secondary binding partners such as linkers or affinity reagents such as an antibody. Indirect binding refers to immobilization of the analyte recognition coating or element to a waveguide. Analyte recognition element can form an analyte capture complex and become attached to the analyte capture surface on the waveguide.

Moieties useful as an analyte recognition coating or element in the invention include biochemical, organic chemical or inorganic chemical molecular species and can be derived by natural, synthetic or recombinant methods. Such moieties include, for example, macromolecules such as polypeptides, nucleic acids, carbohydrate and lipid. Specific examples of polypeptides that can be used as an analyte recognition coating or element include, for example, an antibody, an antigen target for an antibody analyte, receptor, including a cell receptor, binding protein, a ligand or other affinity reagent to the target analyte. Specific examples of nucleic acids that can be used as an analyte recognition coating or element include, for example, DNA, cDNA, or RNA of any length that allow sufficient binding specificity. Accordingly, both polynucleotides an oligonucleotides can be employed as an analyte recognition coating or element of the invention. Other specific examples of an analyte recognition coating or element include, for example, gangilioside, aptamer, ribozyme, enzyme, or antibiotic or other chemical compound. Analyte recognition coatings or elements can also include, for example, biological particles such as a cell, cell fragment, virus, bacteriophage or tissue. Analyte recognition coatings or elements can additionally include, for example, chemical linkers or other chemical moieties that can be attached to a waveguide and which exhibit selective binding activity toward a target analyte. Attachment to a waveguide can be performed by, for example, covalent or non-covalent interactions and can be reversible or essentially irreversible. Those moieties useful as an analyte recognition coating or element can similarly be employed as an secondary binding partner so long as the secondary binding partner recognizes the analyte recognition coating or element rather than the target analyte. Specific examples an affinity binding reagent useful as a secondary binding partner is avidin, or streptavidin, or protein A where the analyte recognition coating or element is conjugated with biotin or is an antibody, respectively. Similarly, selective binding of an analyte recognition coatings or element to a target analyte also can be performed by, for example, covalent or non-covalent interactions. Specific examples of a biochemical analyte recognition coating or element is an antibody. A specific example of a chemical analyte recognition coating or element is a photoactivatable linker. Other analyte recognition coatings or elements that can be attached to a waveguide and which exhibit selective binding to a target analyte are known in the art and can be employed in the device, apparatus or methods of the invention given the teachings and guidance provided herein.

As used herein, the term “radiation power” is intended to mean the amount of energy associated with the reference radiated in one second. Therefore, the term radiation power when used in reference to a measurement as a function of radiation wavelength refers to the amount of radiation energy collected by the detector per second. Similarly, the term “instantaneous radiation power” is intended to mean the amount of energy associated with the radiation in a short sampling time period. The term instantaneous radiation power is used in reference to the amount of radiation energy collected by the detector in a short sampling time period.

As used herein, the term “plurality” is intended to mean two or more referenced signals. Therefore, the term as it is used herein refers to a population of two or more different signals. A plurality can be small or large depending on the design of the apparatus or need of the user. Small pluralities can include, for example, sizes of 2, 3, 4, 5, 6, 7, 8, 9 or 10 or more different signals. Large populations can include, for example, a composite number greater than about 12 or more different signals including tens or hundreds of different signals. Similarly, when used in reference to molecular species or components of a device or apparatus of the invention, the term “plurality” as it is used herein refers to two or more molecules, species or units of the referenced entity.

As used herein, the term “emission detection reagent” is intended to mean a molecule or a material that can emit a specific or characteristic optical or electromagnetic signal, including, for example, selectively scatter, reflect, transmit or emitted electromagnetic radiation. Some emissive detection reagents known in the art can be luminescent, fluorescent or phosphorescent material. The term “luminescence” when used in reference to an emission detection reagent of the invention is intended to mean production of electromagnetic radiation by a chemical or biochemical that is used as or produced by a detection reagent. A luminescent detection reagent can include, for example, luciferase. The term “chemiluminescent” refers to the production of light when the excitation energy derives from a chemical reaction, in contrast to the absorption of photons, in fluorescence. Bioluminescent refers to a subset of chemiluminescence, where the light is produced by biochemical reaction, such as from fireflys, bacteria and other organisms. Specific examples of organisms exhibiting bioluminescence include, for example, Vibrio fischeri, dinoflagellates and sea-fishes. A specific example of bioluminescence is the production of light by a firefly where the substrate Luciferin combines with the enzyme Luciferase and reactants ATP (adenosine triphosphate) and oxygen.

The term “fluorescence” when used in reference to an emission detection reagent of the invention is intended to mean light emission following absorption of energy from an external source of light. Fluorescent emission can be from a chemical or biochemical used as or produced by a detection reagent. The wavelength that is emitted is longer than the wavelength that is absorbed. Specific examples of fluorescent materials include colored dyes such as Cy-3, Cy-5, Alexa Fluor, green fluorescent protein (GFP), silicon nanoparticles, quantum dots, and a diverse collection of other materials well known in the art.

The term “phosphorescence” as it is used herein in reference to an emission detection reagent, is intended to refer to similar phenomenon as fluorescence except that the excited product is relatively more stable. Accordingly, the time until energy is released is longer compared to fluorescence, resulting in a glow after the excitation light has been removed. Phosphorescent emission also can be from a chemical or biochemical used as or produced by a detection reagent. Luminescence, fluorescence and phosphorescence and detection methods employing these phenomenon are known in the art and can be found described, for example, at the url lifesci.ucsb.edu/˜biolum/myth.html.

Other electromagnetic emission detection reagents include colloidal gold, colloidal silver, other colloidal metal plasmon resonant particles, grating particles, photonic crystals and the like. These as well as others are well known in the art and can similarly be employed in the apparatus or methods of the invention given the teachings and guidance provided herein.

The flow-though chemical and biological sensor of the present invention can be used, for example, to detect a wide range of biological, biochemical or chemical analytes. The flow-through chemical or biological sensor of the invention also can be used, for example, to detect one or more of many different analytes in a variety of different formats including, for example, serial, parallel or multiplex formats. Analytes to be detected can include, for example, DNA, RNA, proteins, toxins, bacteria, spores, oocysts, cells, cell fragments, viruses, antibodies, polysaccharides, tumor markers, tissue, food, organic and inorganic compounds, that can be present in or placed into a liquid medium such as water, buffer, serum, whole blood, urine, sweat, sputum, saliva, milk, juices, etc. Similarly, analytes in air and solid samples can also be detected using the sensor or methods of the present invention.

Sample preparation can additionally be employed in conjunction with the apparatus and methods of the invention. Those skilled in the art will know which preparatory procedures are useful given the sample and the analyte to be tested. Specific examples of three sample preparations are provided below for illustrative purposes. Briefly, air samples can be prepared, for example, prior to analysis by bubbling air through a liquid, by use of wet wall cyclone aerosol collector or by electrostatic aerosol collector as well as others well known in the art followed by testing the liquid. A solid sample can be prepared, for example, prior to analysis by dissolving the sample in a liquid solution or mixing or homogenizing it in a liquid. A solid sample also can be embedded in a matrix with subsequent processing into a suitable liquid or particulate suspension. Preparation of the matrix that an analyte can be embedded is known in the art and can differ depending on the matrix and the analyte. For an example, the following procedure can be used to prepare ground beef samples to detect E. coli O157 (see, for example, D. R. DeMarco and D. V. Lim, Detection of Escherichia coli O157:H7 in 10- and 25-gram ground beef samples using an evanescent wave sensor with silica and polystyrene waveguides. J. Food Protection 65, 596-602 (2002). Twenty five gram samples of commercially-purchased ground beef in sterile, plastic conical tubes can be homogenized with twenty-five ml of buffer. The homogenized sample will be centrifuged for 5 minutes at 4° C. A middle layer containing pathogen can be collected and transferred to a sterile tube, and mixed by vortex. The obtained sample is suitable for use in the sensor and methods of the invention.

Additionally, preparatory procedures suitable for the testing of pathogens in a liquid can additionally include a filtering, concentration or centrifugation step or combinations of these steps. Those skilled in the art will understand given the teachings and guidance provided herein which preparatory step is beneficial to include depending on the nature and quantity of the liquid and sample analyte. For example, it can be beneficial to remove large particles in the liquid, as well as other contents that could interfere with the sensor's operation. To detect low concentrations of pathogens in the liquid, the liquid can be concentrated and the concentrated liquid used for the sensor assay.

For waveguide sensors, the waveguide can be coated with an appropriate molecular recognition species, also referred to herein as an analyte recognition coating or analyte recognition element. Where the capture configuration is in solution phase for initial analyte binding as described previously, the waveguide can be coated with a secondary binding partner. Such coatings, elements or secondary binding partners can include, for example, a protein (e.g., antibody, antibiotic, an antigen target for an antibody analyte, cell receptor protein, avidin), a nuclear acid or related to nucleic acid (e.g., oligonucleotide, DNA, cDNA and RNA), polysaccharide, monosaccharide, oligosaccharide, aptamers, ribozymes, enzymes, ligands, cell and cell fragment as well as other biological particles. This molecular recognition species will serve to capture the analyte on the waveguide when the assay is performed. The prepared waveguides can be stored until use when the assay is performed or used immediately after functionalization with a recognition species.

The presence of the analyte can be detected, for example, via electromagnetic radiation. All wavelengths within the electromagnetic spectrum that can transmit in the waveguide can be used to specifically detect an analyte using, for example, an emission detection reagent. Useful detection spectrum includes, for example, the visible spectrum, emitted by a fluorescent, phosphorescent or luminescent detection reagent or label attached to, for example, a secondary molecular recognition species and infrared spectrum. The labeled secondary molecular recognition species can be any labeled species that recognize and bind to the captured analyte or to the complex formed by the analyte bound by the primary molecular recognition species such as an analyte recognition coating or element.

In addition, binding and detection methods and other than those described above and below are known in the art. Such other methods and formats are equally applicable in the sensor apparatus or methods of the invention. The apparatus and methods of the invention include the capture of an analyte by an analyte recognition coating or element. Capture can be accomplished by, for example, any affinity binding means that is specific for the analyte of interest. For example, binding formats applicable for use in the invention include direct binding of the analyte by the analyte recognition element or indirect binding by, for example, an intermediate affinity binding reagent. Binding and detection also can be performed in a sandwich format in which the analyte is bound between an analyte recognition coating and a detection reagent. As described previously, capture of the analyte can be via solution or solid phase configurations with the analyte recognition coating or element and then bound by a secondary binding partner to a waveguide. Other formats well known to those skilled in the art also can be employed in the apparatus and methods of the invention.

Further, the apparatus and methods of the invention include the detection of bound analyte by an emission detection reagent. Various emission detection reagents well known to those skilled in the art can be employed in the sensor apparatus and methods of the invention. Such emission detection reagents include, for example, luminescent, fluorescent and phosphorescent emission detection reagents, all of which can be employed with any of the various binding methods or formats described herein or well known to those skilled in the art. Additionally, such detection reagents can be employed in modes that include direct binding to an analyte or an analyte bound to a recognition coating. Alternatively, emission detection reagents can be employed in modes that include indirect binding to an analyte or an analyte bound to a recognition coating. Further, the binding and detection methods and formats for analyzing also can include methods such as FRET (fluorescence resonance energy transfer) where an optical signal is generated following a change in proximity of the fluorescent detection reagent from the quencher following binding of analyte. A change in proximity can include, for example, a release of the emission detection reagent such as by cleavage with a protease analyte, or a change in conformation due to analyte binding.

The binding or detection methods or formats are well known to those skilled in the art and can be employed in the apparatus of the invention. Similarly, other well known binding or detection methods or binding or detection formats also can be employed in the apparatus or methods of the invention. Given the teachings and guidance provided herein, those skilled in the art will understand that any of the various binding or detection methods or formats known in the art can be used in conjunction with the methods or formats described herein. Similarly, given the teachings and guidance provided herein, those skilled in the art will understand that the various binding or detection methods or formats can be substituted or used in various combinations with the methods and formats exemplified herein.

The invention provides a mixing flow apparatus. The mixing flow apparatus consists of a waveguide and a mixing flow chamber; the waveguide having an appropriate index of refraction material for propagation of a radiation signal, and the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet. The mixing flow chamber surface can be positioned to disrupt flow regularity by, for example, structural or spatial configurations. The mixing flow chamber surface also can be positioned to disrupt flow regularity by, for example, inclusion of specific shapes or being activatable. Shapes include, for example, physical protrusions as well orifices that allow injection of gases, vapors and the like that disrupt flow directly or that generate bubbles which disrupt flow.

Also provided is a detection apparatus. The detection apparatus consists of a waveguide, a mixing flow chamber and a radiation detector; the waveguide having an appropriate index of refraction material for propagation of a radiation signal; the mixing flow chamber having a body forming a flow chamber with an inlet, an outlet, a radiation transmissive wall and a surface positioned to disrupt flow regularity of a sample fluid, the body of the mixing flow chamber surrounding at least a portion of the waveguide, wherein constituents of a sample fluid entering the inlet are mixed by disruption of sample fluid flow regularity prior to discharge at the outlet, and the radiation detector being disposed facing the direction of oncoming propagated signal from the waveguide. The mixing flow chamber surface can be positioned to disrupt flow regularity by, for example, structural or spatial configurations. The mixing flow chamber surface also can be positioned to disrupt flow regularity by, for example, inclusion of specific shapes or being activatable.

A mixing flow apparatus of the invention consists of a waveguide and a mixing flow chamber. The apparatus can be used alone as a mixing device or for the detection of analytes with inherent optical emissions. In the latter example, the mixing flow apparatus can be coupled, for example, to a detector for measuring analyte emissions. Alternatively, qualitative observation can be used when the emission intensity is sufficiently strong. Additionally, the mixing flow apparatus can be combined, for example, with a radiation source or a detection device to produce a sensor. The mixing flow chamber or cartridge can be a stand alone cartridge or part of a larger cartridge. Specific examples of a mixing flow chamber or cartridge include those shown in the figures and described further below as well as micro chips and microfluidic chips. The various embodiments of the mixing flow apparatus or the apparatus combined with other sensor hardware for detection of incident radiation are exemplified below.

For example, radiation or detection hardware of the sensor can include an instrument to control and perform an assay and a chamber or vessel in which the assay takes place including, for example, any stirring or mixing of the reagents and analyte that results in the capture and identification of the analyte. This chamber or vessel can be affixed to or detachable from the instrument and can be a reusable, rechargeable, or disposable cartridge. This chamber or vessel is also referred hereafter in its various forms as a mixing flow cartridge. A mixing flow cartridge consists of at least one mixing flow chamber and at least one waveguide. The mixing flow cartridge can be re-usable for a number of times. Reuse of the mixing flow cartridge is particularly useful in instances where initial test results are negative. The sensor instrument also can include, for example, radiation illumination member(s), radiation detector member(s) (such as photodiodes, CCDs, photomultiplier tubes (PMTs), position sensitive PMTs, CMOS arrays, spectrometers, etc.), a fluid handling member (such as pumps, valves, switches, meters, etc), electronics member (such as circuits, displays, timers, etc.) and software programs.

The fluid flow in the mixing flow chamber is designed to improve the capture of the analyte by the waveguide by passively or actively stirring the sample to enable constituents of the sample to come in contact with the analyte capture surface. Exemplary embodiments of passive mixing of the analyte include, for example, the inclusion of an undulating shape of the mixing flow chamber wall. This undulating shape can cause the fluid in the mixing flow chamber to move about in a turbulent manner as it flows from inlet to the outlet. A static waveguide inside the body of the mixing flow chamber also can act as a mixing element, creating turbulence in the sample. Additionally, the waveguide inside the body of the mixing flow chamber can be, for example, attached to the mixing flow chamber on one end and the other end is allowed to move.

Exemplary embodiments of active mixing of the analyte can include, for example, unattached or attached members of similar or different material placed inside the mixing flow chamber. These members can be allowed to move inside the body of the mixing flow chamber and also can be actuated by mechanical, thermal, electrical or magnetic forces. Additionally, for example, sample can be pumped into a mixing flow chamber from different inlets and pumped out of the mixing flow chamber from different outlets at the same or at different times. The flow direction can be periodically reversed. The pumping speed also can be modulated.

With respect to the physical structure of a mixing flow chamber or cartridge, in one specific embodiment a mixing flow chamber can consist of a fluid sample mixing flow chamber having a body, at least one waveguide member. As stated previously, the waveguide can be, for example, connected to the mixing flow chamber. Alternatively, the mixing flow chamber also can include, for example, a waveguide not connected to the mixing flow chamber or a mixing flow chamber can include multiple waveguides, all connected to the mixing flow chamber, some connected and some unconnected to the mixing flow chamber or all unconnected to the mixing flow chamber. A mixing flow chamber also can include, for example, chambers containing reagents and a chamber to be filled with sample fluid. This embodiment of a mixing chamber includes a first end and a second end, side walls, a clear top surface, a bottom surface, at least one inlet, and at least one outlet. The body of the chamber extends outward from the waveguide member and is spaced therefrom so as to allow a fluid to flow between the inlet and the outlet.

The waveguide member can be coated with analyte capture elements. Assays can be performed to capture the analyte, and the analyte can be tagged with an emission detection reagent or labels. Excitation light impinges on the emission detection reagent to cause it to produce light. The waveguide, capturing a portion of the emission light along with some excitation light and propagating them to one end. The light emerges from the waveguide and passes through lens, filters or grating system before detection by an optical or infrared detector.

The body of the mixing flow chamber can consist of, for example, one clear element through which the excitation light enters the mixing flow chamber. This clear element can have flat top and bottom surfaces to provide uniform illumination along the long direction. This clear element can have curved surfaces to focus the excitation light on to the waveguide. This clear element can also serve as a waveguide. The ends of this clear element can be coated with reflective material. Some parts of the sides or other areas of this clear element can be coated with reflective and/or light absorbing material. Additionally, when the clear element of the body is not the waveguide, some parts of this clear element can be coated with light absorbing material.

One or more sides of the mixing flow chamber can have undulating surfaces that vary in the long direction and that serve to stir the fluid as it flows through the mixing flow chamber, while other portions of the surface can be smooth in the long direction. The undulating shaped surfaces can be on one side, two sides or all sides of the mixing flow chamber. Some parts of the undulating and smooth surfaces can have light absorbing properties. Some parts of the undulating and smooth surfaces can have reflective properties. Some parts of the surface can be clear. Undulating walls can have any shape, as long as they function to mix the sample fluid and minimize fluid trapping. An undulating shape can be periodic in the long direction, for example.

In another specific embodiment of the invention, no surface of the mixing flow chamber has undulating walls. The surfaces of the mixing flow chamber are smooth and can be flat or have uniform curvature. The mixing can be performed, for example, by flow over stationary waveguides inside the mixing flow chamber, by waveguide motion inside the mixing flow chamber actuated externally by the waveguide motion induced by the flow over the waveguide, by motion of embedded elements inside the mixing flow chamber actuated externally by electric or magnetic forces, or by temporally or persistent modulated pumping action of the fluid.

The material of the mixing flow chamber wall can be different or the same as the waveguide. The mixing flow chamber can have at least one inlet and at least one outlet.

In another specific embodiment of the invention, the mixing flow chamber can have multiple mixing flow chambers each with at least one each of waveguide, inlet and outlet.

The body of the mixing flow chamber can be made of any material compatible with the sample fluid and assay reagents. Generally, the body of the mixing flow chamber is made of a polymer that can be manufactured by, for example, injection molding, such as polymethylmethacrylate, polycarbonate, or polystyrene. The body of the mixing flow chamber forms a tight seal to prevent loss of sample fluid. The body of the mixing flow chamber can be either rigid or elastic. Materials for all parts of the body of the mixing flow chamber should be compatible with the analyte and the assay reagents. Given the teachings and guidance provided herein, those skilled in the art will know, or can readily determine those material having compatibility with the analyte binding and detection methods described herein.

With regard to the waveguide, radiation from emission detection reagent attached to a higher index of refraction waveguide than its surroundings is partially radiated into the waveguide and partially into the surroundings. See Jin Au Kong, Electromagnetic Wave Theory (First Edition, John Wiley & Sons, Inc., New York, 1975; Second Edition, John Wiley & Sons, Inc., New York, 1990) and Cha-Mei Tang, IEEE Transactions On Antenna And Propagation, AP-27 (5), 665-670 (1979). In the context of the apparatuses of the invention, the higher index of refraction material for propagation of an emitted signal is referred to herein as a waveguide. The waveguide provides the ability to direct the emitted signal into the waveguide and to the detector.

The waveguide can be, for example, one of the elements that constitute the sides of the mixing flow chamber, or it can be suspended in the middle of the mixing flow chamber. The waveguide can have any shape. Generally, the waveguide is elongated in one dimension. The surface of the waveguide should be optically smooth to provide low loss of the optical signal.

The shape of the cross section can vary so as long as it remains a medium that can propagate an optical signal for at least a short distance, such as the distance from signal emission along the waveguide to the exit end of the waveguide to the detector. This distance also can include the entire length of a waveguide. For example, some of cross sectional shapes can be circles, ovals, ellipses, squares, rectangles, diamonds, polygons rings, or other shapes that can propagate emitted radiation signal from captured analyte to a detector. Accordingly, a waveguide does not need to be straight in the long direction. It can have sections that include arcs, loops, oscillations, so long as it facilitates propagation of an emitted radiation signal from captured analyte to a detector.

A waveguide can be made of any material, for example, that transmits light at both the excitation wavelength and the signal emission wavelength. A waveguide can consist of a single material or consist of a composite of two or more different materials. The composition of waveguide materials can vary, for example, in the long direction as well as in the transverse direction. Different sections can have different materials. Generally, the waveguide can be an inorganic glass or a solid such as a polymer (e.g., a plastic such as polystyrene). The waveguide can have multimode or single mode optical properties.

The waveguide can be coated with reflective material on the surfaces of some of the transverse direction, or on one end of the waveguide. The reflective coating can be any material that reflects light at the excitation wavelength at some parts of the waveguide, and the coating can reflect light at the emitted signal wavelength at some parts of the waveguide, or both. The reflective coating can also be any material that reflects both the excitation and emission wavelength. Generally, a reflective coating includes a reflective metal, such as aluminum, silver, gold, chromium, platinum, rhodium, or mixtures thereof. More often, a reflective metal is aluminum, silver, or gold. Additionally, the reflective coating can consist of multiple layers, such as dichroic mirror, or reflective material and bonding material.

The reflective coating can be applied to the surface of the waveguide in any manner known in the art for such procedures. Vacuum evaporation deposition of the reflective coating on glass and plastic substrates is one exemplary method. Lithography patterning technique also can be used. Electroless deposition is yet a further exemplary method.

Specific examples of waveguides include a round optical fiber having transmission properties. The round optical fiber can be coated on one side with reflective coating. When used as a waveguide, one laser source will be able to provide improved uniformity of illumination. Rectangular optical fiber coated with reflective material on two opposite sides can provide uniform illumination and good signal transmission. Further, for example, capillary tubes can be used both as a mixing flow chamber and waveguide. Capillary tubes can be coated with reflecting material on a portion of the exterior surface to improve the illumination of the analyte capture surface inside the capillary tube.

The waveguide shape and features can vary along the long axis. Some common changes in features are the dimensions of the waveguide, abrupt transition in shape, or smooth transition in shape or changes in coatings. For example, the cross sectional size can vary from a circle of larger diameter to a smaller diameter. For example, the cross sectional shape can vary from a polygon to a circle.

In addition, the present invention allows the attachment to the waveguide of other optical elements. Such other optical elements can include, for example, lenses or optical filters.

The mixing flow or detection apparatuses of the invention can be used for single or multiple analyte detection. For example, the apparatuses and methods of the invention allow for detection of a single analyte or the simultaneous detection a multiple analytes on a single waveguide or on multiple waveguides, independently or simultaneously. The optically clear surface of the waveguide inside the mixing flow cartridge serves to capture the analyte to be measured. The amount of surface area needed for detection depends on the desired detectable concentration level. A range of the analyte capture surface area can vary from 0.01 μm² to many cm².

In one specific embodiment, multiple analyte detection can be achieved by patterning the waveguide in sections, each with a different analyte capture surfaces sensitive to a specific analyte.

In another specific embodiment, the waveguide surface is simultaneously coated with different analyte capture elements. As sample flows through the mixing flow chamber, multiple analytes in question can be simultaneously captured along the whole length of the waveguide.

In a further specific embodiment, multiple analyte detection can be achieved with a sensor having multiple mixing flow chambers. In this embodiment, each of these mixing flow chambers contains at least one waveguide that is coated with an analyte capture surface.

In still a further specific embodiment, more than one waveguide can be used to detect the same analyte. This method can be used to increase the analyte capture surface area or to increase the mixing of the fluid.

A mixing flow apparatus can be configured as a detection or as a signal detection apparatus. Such detection or signal detection apparatuses can consists of, for example, (1) one or more light sources to illuminate (excite) the emission detection reagent to produce a signal light, (2) optical system, (3) a detector system to capture the emitted signal light, (4) fluid handling system, (5) data acquisition, signal analysis and data output. For detection of analytes having inherent optical properties, such as chemiluminescent labels, the illumination source can be omitted or unused in the apparatus.

One component of the instrument is the radiation illumination member, consisting of light source(s) and optics. For some applications, such as colloidal gold and silver, the excitation light source can be a broad spectrum source while in other applications, the excitation light source can be a narrow spectrum. Some waveguides can be better illuminated using multiple light sources. In some multiple-analyte applications, for example, with more than one fluorescent label on the same waveguide, some labels can require one or more narrow band excitation light sources, while other labels, such as quantum dots, can require a single broadband excitation light source for all emission wavelengths. Lenses, filters, and other optical devices can be needed to achieve the desirable illumination.

Excitation light source in the present invention can use any light source using any of various methods well know in the art. Exemplary sources include, lasers, light emitting diodes (LEDs), and broadband light sources.

Briefly, light from a laser has the property of coherence and potentially high power, narrow band in wavelength, that can be turned into a wide collimated beam, a cone beam or a fan beam with lenses. Coherence and high power provide larger power density. Narrow band is desirable for organic dyes. Any kind of laser can be used in the apparatuses and methods of the invention. Diode lasers are commonly available, compact and relative low cost.

Light Emitting Diodes (LEDs) produce incoherent light. LEDs are inexpensive and compact and therefore beneficial for some applications. Alternatively, an addressable multiple-element array of optical sources, such as LEDs, can be used to sequentially probe each patterned region of the waveguide. This multiple element array of optical sources provides a particularly low cost technique, having the advantage of no moving parts, and providing more flexibility than stepped or oscillated excitation light, because LEDs or groups of LEDs would be addressable in any arbitrary temporal or spatial sequence.

Broadband incoherent light sources including, for example, incandescent lamps, xenon lamps, mercury lamps and arc lamps also are useful in the apparatuses of the invention. For example, broadband ultra violet (UV) sources can be useful for illuminating quantum dot labels.

A wide variety of excitation light source configurations are possible for using in the radiation illumination member. The selection among alternatives will depend, in part, on the type of recognition element patterning on the waveguide.

In this invention, the temporal mode of radiation illumination and radiation detection can include, for example, a variety of methods and variations. Specific examples of such modes include instantaneous signal, time averaged instantaneous signal, time integrated partial signal, time integrated continuous whole signal, frequency modulated signal, or other variations or combinations thereof. The temporal mode of illumination and detection is related to the method of spatial illumination of the excitation light, the fluorescent labels, the waveguide geometry, the number of analytes to be detected, the concentration level of the analyte, and the desired sensitivity of the detection.

Excitation light source can impinge on the emission detection reagent of one or more analytes during the entire period of detection of each analyte. The excitation light source can be modulated or “chopped” as a means to eliminate interference from ambient light. Demodulation of the resulting emitted signal, such as with a lock-in amplifier, can then reduce background interference. Such modulation can not be required, if ambient light is eliminated by proper optical isolation or shielding.

One method of illumination is for the excitation light source to emanate from a wide or diffused area, and to illuminate the entire analyte recognition surface of the waveguide(s) from one or more directions. Advantages of this unfocused or diffused area of illumination method include: (1) it would illuminate substantially the entire analyte sensing area on one or more waveguides, (2) it minimizes alignment procedures, since the illumination areas is larger than the waveguide areas.

An alternate method of illumination is for the excitation light source to emanate from a point source or to be focused to a point source, and thence illuminate the analyte-sensing area on the waveguide. This method of illumination can use focused or collimated light from a laser or other source and can illuminate a portion of the waveguide. Advantages' of this focused or point source method include: (1) greater excitation light intensity; (2) ability to control and manipulate the angular distribution of the excitation light; (3) the potential to use high sensitivity, background- and noise-rejecting electronic signal processing methods (e.g., modulation and demodulation); and (4) possibility to reduce cross talk from other analytes and nearby waveguides.

One or more excitation light sources can be used sequentially or simultaneously to provide different illumination wavelengths and/or to provide different spatial and temporal coverage. The angle of incidence of the excitation light can be perpendicular to the incident surface of the waveguide, perpendicular to the length of the waveguide, or at one or more angles in relation to the surface of the waveguide. The optimal angle of illumination can be selected so as to reduce the background noise resulting from excitation light or to enhance any other desirable characteristics of the sensor. The excitation light can be collimated, non-collimated, point source, multiple point sources, diffused source or broad area unfocused source. The angle of illumination is not limited to excitation perpendicular to the surface of the waveguide.

An optimal angle of illumination is dependent on the size and shape of the waveguide and the desired detection limit. Long waveguides can reduce collected excitation light at the detector because each time the excitation light reflects on a boundary of the waveguide, part of the excitation light is lost due to transmission out of the waveguide. The loss is largest at the perpendicular angle. The excitation light can also be in the form of evanescent wave with the light input at the end of the optical fiber.

A radiation detection device can be placed where light exits from the waveguide in order to detect the signal produced by the label(s). The detector assembly can consist of an optical system in addition to the radiation detection device.

Emission signals produced by the labels can be detected by a variety of different detectors, such as photodiodes, one-dimensional charge-coupled device (CCD) arrays, two-dimensional CCD arrays, photo-multiplier tubes (PMT), position sensitive PMTs, CMOS image arrays, spectrometers, etc. The PMT should preferably be chosen to have maximum sensitivity in the region of radiation of the labels and should preferably be provided with a filter blocking the light emitted by the source radiation. One or more detectors can be used.

The emission signal produced by the labels can be detected (1) as a total power independent of the frequency or position, (2) as a total power as a function of position independent of the frequency, (3) as power in the frequency spectrum independent of position and (4) as power as a function of position and frequency.

The emission signal produced by the labels can be amplified electronically or using photomultiplier tubes (PMTs). The emission signal produced by the labels can be detected as instantaneous, time averaged or time integrated power. For labels such as quantum dots, which can remain photo stable after exposure to long periods of excitation light sources as compared to organic dyes, integration of the signal over long period of time becomes possible and can be used to improve the sensitivity.

Optics are used to minimize the excitation light entering the detector. Some examples of the embodiments are as follows: (1) use of wavelength dependent filters, (2) use of a grating outside the waveguide to spread the light into a spectrum of wavelength and use only the signal from the emission light wavelength, and/or (3) use of gratings or absorbent coatings on the waveguide surfaces to allow the transmission of emission light and prevent the transmission of excitation light from the waveguide to the detector.

Various lenses, mirrors, and optical filters can be placed between the waveguide and the detector. For example, a linear lens array in registration with the waveguides can be used. Other options include the use of a pair of linear Gradient-Index (GRIN) lens arrays configured to provide a quasi-collimated region between the arrays for insertion of an interference filter, and an array of cylindrical lenses. Alternatively, optical filters can be directly butt-coupled to the waveguide or to the detector, or both.

The apparatuses of the invention can be automated to include a fluid handling member, which consists of valves, pumps, switches and reagent chambers. The sensor can be constructed with valves, pumps, switches, and reagent chambers as part of the instrument using conventional off-the-shelf components, or some or all these elements can be constructed as part of the mixing flow cartridge.

Fluid flow can be achieved manually with a syringe or other vacuum or pressure device, or automated using a pneumatic, peristaltic, or microfabricated pumps designed to move the solutions inside the mixing flow chamber. A non-optical filter can be placed at the inlet of the mixing flow chamber in order to prevent undesirable particles from entering the mixing flow chamber. The sample can be recirculated through the mixing flow chamber to increase the chance of capture. Samples can enter from more than one inlet and exit from more than one outlet. The flow into each inlet and out of each outlet can individually and temporally modulated. The flow direction can be reversed, such that the inlet can become the outlet for certain periods of time.

The types of assays that can be performed include, for example, (1) a competitive assay (wherein labeled and unlabeled analyte compete for open binding sites), (2) a displacement assay (wherein unlabeled sample analyte dissociates bound labeled analyte or molecular recognition species on a waveguide that has been previously coated with bound labeled analyte), (3) a sandwich assay (wherein sample analyte binds to a primary molecular recognition species on the waveguide surface, and a labeled secondary molecular species binds to the immobilized analyte or the immobilized analyte/primary molecular species complex), nucleic acid hybridization assay, (4) Fluorescence Resonance Energy Transfer (FRET) assay (wherein sample analyte causes a change in a recognition species bound on the waveguide to produce a fluorescent signal), (5) chemiluminescence assay (wherein sample analyte causes a chemical or other type of reaction on a recognition species bound on the waveguide to produce a luminescent signal), or any other type of bioaffinity or chemical affinity assay that produces a detectable signal.

A wide variety of analyte recognition elements and methods for attaching them on the waveguides can be used with the present invention. One common feature among the various assays is that the surface of the waveguide is coated with an analyte recognition element. Analyte recognition on the waveguide surface can also be accomplished by means other than the attachment of a molecular recognition species. For example, the analyte capture surface can be formed by coating the waveguide surface with a binding material, such as avidin, a doped or undoped polymer, or sol-gel that exhibits a differential optical response upon exposure to the analyte or an analyte complex including, for example, a combination with an additional label or labels. An example of one such non-biomolecular recognition species is provided in MacCraith, Sensors & Actuators 29(1-3), 51-57 (1995).

Regardless of how analyte recognition is achieved, an emission detection reagent is typically used to generate an optical signal to indicate the presence or absence of the analyte. If a sandwich assay is desired, the labeled secondary molecular recognition species can be any labeled species that recognizes a molecular binding site on the analyte capture complex, immobilized analyte or the immobilized molecular recognition species/bound analyte complex.

In the present invention, typical methods for attaching molecular recognition species to surfaces include covalent binding, physisorption, biotin-avidin binding (such as described in Bhatia et al., Use of Thiol-Terminal Silanes and Heterobifunctional Crosslinkers for Immobilization of Antibodies on Silica Surfaces, Anal. Biochem. 178 (2): 408-413, May 1 (1989); Rowe et al., An array Immunosensor for Simultaneous Detection of Clinical Analytes, Anal. Chem. 71 (2), 433-439 Jan. 15, 1999; Conrad et al., U.S. Pat. No. 5,736,257; Conrad et al., SPIE, 2978, 12-27 (1997); Wadkins et al., Biosensors & Bioelectronics 13 (3-2): 407-415 (1998); Martin et al., Micro Total Analysis Systems (Kluwer Academic Publishers, Netherlands, 1998 p. 27), or modification of the surface with thio-terminated silane/heterobifunctional crosslinker as in Eigler et al. [sic], U.S. Pat. No. 5,077,210 issued Dec. 31, 1991, or the use of APTES/NHS-Maleimide bifunctional linker/Thiol modified polyethylene glycol (see Soon Jin Oh et al., Langmuir 18, 1764-69 (2002)). The immobilization of molecular recognition species to the waveguide can also use polyamidoamine (PAMAM) dendrimers (See R. Yin et al., Dendrimer-Based Alert Ticket: A Novel-Biodevice for Bio-Agent Detection, Polymeric Materials: Science & Engineering 84, 856-857 (2001)). Attachment of analyte recognition species can also be achieved by photolithographic method. Alternatively, attachment of molecular recognition species to the waveguide surface can use commercial products such as dendrimer based self assembled monolayer (SensoPath Technologies, Inc., Boseman, Mont.).

Furthermore, in the present invention fluorescent dyes, fluorescent nanoparticles, quantum dots, colloidal gold, colloidal metal plasmon resonant particles, Fluorescence Resonance Energy Transfer (FRET), chemiluminescence and other fluorescent sources can be used to produce the optical signal produced by the capture complex or the analyte/capture complex on the analyte capture surface of the waveguide. In other words, the present invention is not limited by the source or type of assay components.

In one embodiment of the invention, the mixing flow cartridge, containing the waveguide coated with the molecular recognition species, can be stored for a period of time before being used. In another embodiment of the invention, the mixing flow cartridge, containing the waveguide coated with the molecular recognition species and an appropriate labeled or unlabeled analyte/molecular recognition species, can be stored for a period of time before being used in a displacement assay.

Finally, it should be kept in mind that the waveguides on which analyte capture species are coated can be, for example, used more than once. Thus, after detection and analysis, the waveguide can be exposed to an appropriate chemical, biological, or optical, or other treatment as known in the art that is capable of removing the analyte or otherwise restoring the original analyte-sensing properties of the molecular recognition species.

Molecular recognition on the analyte capture surface can also be accomplished by means other than the attachment of a molecular recognition species. For example, the analyte capture surface can be formed by coating a surface of the waveguide with avidin, a doped or undoped polymer or sol-gel that exhibits a differential optical response upon exposure to the analyte or the analyte in combination with an additional label or labels. An example of one such non biomolecular recognition species is provided in MacCraith, B D., Sensors and Actuators B., 29 (1-3): 51-57 October 1995, the entirety of which is incorporated herein for all purposes. The analyte capture surface of the waveguide can be prepared, for example, after the complete construction of the mixing flow cartridge or prepared before the final assembly.

Generally, the space between waveguide member and mixing flow chamber walls can have a dimension of few tens of microns to a few millimeters. The waveguide can have a cross sectional dimension of few microns to few millimeters and have a long dimension of few hundreds of microns to tens of centimeters.

One or more combinations of the sample flow can be employed. For example, single pass, where the sample enters the inlet and exits from the outlet can be employed. Alternatively, recirculating flow, where the sample enters the inlet and exits from the outlet and this process is repeated, improving the percentage of capture over single pass also can be employed. Alternatively, pulsed flow, where for example, the sample flows enters and exits the mixing flow chamber at different velocities at different times creating mixing followed by incubation also can be employed. Additionally, reversible flow, where the sample flows in one direction and the direction reverses so that the inlet becomes outlet and outlet becomes inlet, a useful method when the waveguide layout is not symmetric to the inlet and outlet.

Multiple inlets and outlets can be utilized, including more than one inlet and/or more than one outlet. This configuration can provide a desirable distribution of fluid flow and higher flow rate. Different entrances and exits also can be utilized where, for example, a sample enters different inlets at different times and exits different outlets at different times. Further, multi-analyte testing also can be performed in the apparatuses of the invention. In this embodiment, the same sample can be passed over all the waveguides, each of which can be detecting for a different analyte.

The sensor, in one aspect of the present invention, allows for manual or automated detection of analytes. The instrument format can be a portable kit, a bench top instrument or large high throughput processing systems that can be used to detect and quantify a variety of hazardous substances in numerous sample matrices. The instrument can be used in different types of environments. It allows for rapid and accurate detection of any sort of analyte present in food, water, soil extracts, air extracts, and clinical fluids.

In the following description, in order to facilitate a thorough understanding of the invention and for purposes of explanation and not limitation, specific details are set forth, such as a particular geometry of the sensor. The invention can be practiced, however, in other embodiments that depart from these specific details.

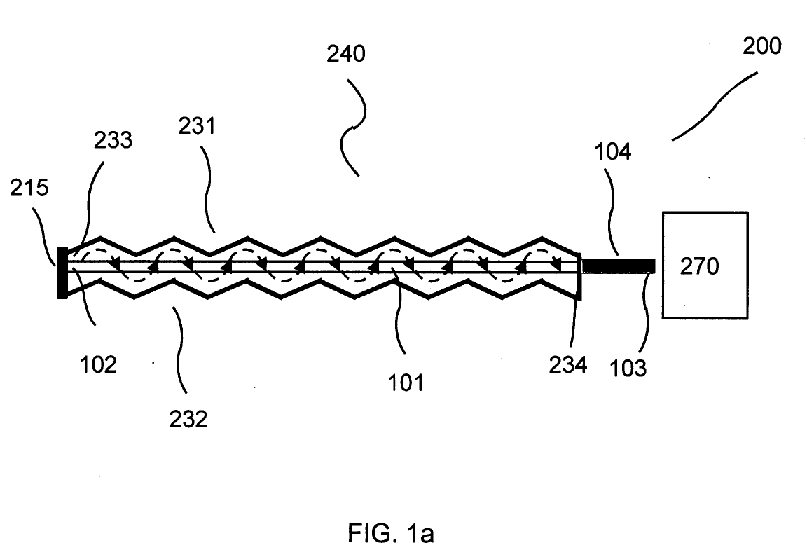

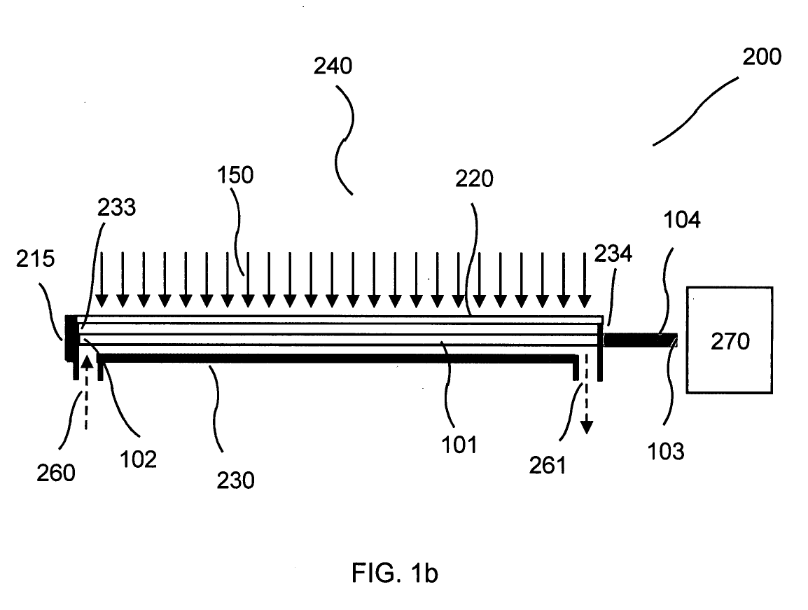

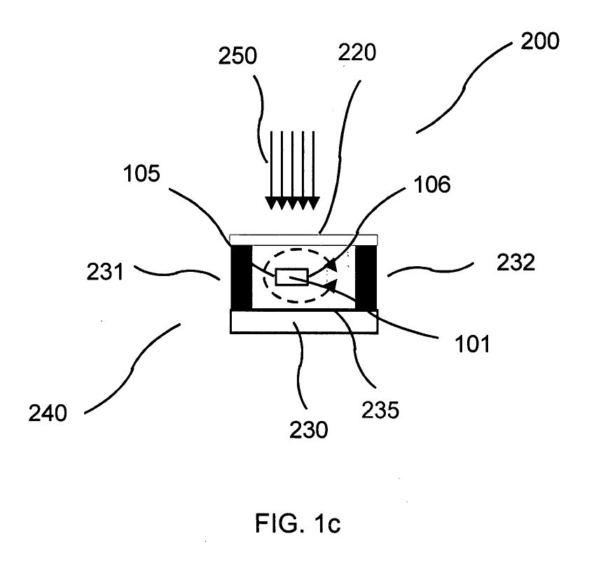

FIGS. 1a, 1b and 1c show the top view, side view and end view of a mixing flow-through sensor according to one embodiment of the invention, respectively. Sensing system 200 consists of a mixing flow chamber 240, waveguide member 101 on which is attached the analyte capture surface, and the detector member 270.

In the embodiment shown in FIG. 1a, waveguide member 101 is an elongated member, adapted to propagate along its length the collected radiation. The waveguide member 101 passes through mixing flow chamber 240, so as to expose substantially all of the waveguide surface to the sample, leaving first end 102 and second end 103 of the waveguide unobscured. A reflective surface 215 can be placed at the end of the waveguide 102. More particularly, mixing flow chamber 240 consists of elongated side bodies 231 and 232 that extends outward from waveguide member 101 and is constructed and arranged to house a portion of the waveguide. The mixing flow chamber 240 also consists of first end 233 and second end 234, and the waveguide member 101 is attached at least to the second end 234. The emission signal exits from the waveguide end 103 and enters the detector member 270.

FIG. 1b shows the side view of the mixing flow chamber 240 and further consists of radiation transmissive surface 220 allowing the excitation light to propagate to the analyte capture surface of the waveguide. The lower border 230 can be clear, black or any other color, or coated with reflective or absorbent material. The mixing flow chamber includes an inlet 260 and an outlet 261 to allow a fluid solution to flow inside the mixing flow chamber between inlet 260 and outlet 261. In another embodiment, the position of inlet 260 and outlet 261 can be reversed. The excitation light 250 is incident on the waveguide surface.

FIG. 1c shows the end view of the sensing system 200, including transparent top boundary 220, the side walls 231 and 232, the bottom wall 230, the waveguide 101 and incident radiation 250.

The walls 231 and 232, as shown in FIG. 1a, are undulated so that they force the liquid sample to flow from one side of the mixing flow chamber to the other side and to go around the waveguide, as shown in FIG. 1c. The waveguide acts as a mixing stick as indicated in FIG. 1c. Specifically, the shape of the walls and the waveguide 101 prevents the flow from being laminar, and allows all of the analyte in the sample to have a chance to come in contact with the analyte capture surface on the waveguide. The motion of the sample to the first order is indicated by the dashed curves in FIGS. 1a and 1c. Depending on the characteristics of the fluid and the flowing conditions, a turbulence regime can be established inside part of the mixing flow chamber. As a result, the interactions between the constitutive elements of the fluid and the analyte capture surface of the waveguide are significantly enhanced. It follows that the mechanical interactions between the elements of the fluid and the surface of waveguide member 101 predominate over diffusion process. As a result, the amount of analyte captured onto the waveguide is increased, while the time required for doing so is decreased. The flow of fluid also deters non-specific binding of other material in the sample to the waveguide and mixing flow chamber.

However, it should be apparent to one skilled in the art to which the invention pertains that alternative shapes of the mixing flow chamber can also be used to carry out the object of the present invention. The mixing flow chamber does not need to be rectangular. It can be any shape that causes the fluid to mix. The undulation of the walls, for example, (a) can be periodic or non periodic, (b) can have sharp corners as shown in FIG. 1a or smooth curves (c) can vary only in two coordinates called two-dimensional as shown in FIG. 1a or can vary in three coordinates called three-dimensional, (d) can have undulation on one wall, two walls, three walls, or all sides (e) can have a different undulating pattern on each wall, and any other variation or combination of these features. The shape of the walls should be chosen such that the fluid sample is forced to flow around the waveguide. The shape of the walls should not result in pockets of stagnation.

Mixing flow chamber 240 in FIGS. 1a-c can be made from any material chemically compatible with the analyte and the fluid solution being assayed. In addition, the mixing flow chamber can be either rigid or elastic, and can be a single material or a composite or multilayer structure. The radiation transmissive top surface 220 is preferably very low loss, fabricated from such materials as glass, plastics such as polycarbonate, polystyrene, polyacrylic, or other clear material. The outside surface of the mixing flow chamber wall 220 can be coated with non-reflective coating to increase the light impinging on the analyte capture surface of the waveguide. The side walls 231 and 232 preferably are made with radiation absorbing material with the property of black color and non reflective, including but not limited to plastics or any other easily molded materials.

In addition, some of the walls of the mixing flow chamber 240 can be coated with reflective material. For example, the surface 236 on wall 230 of FIG. 1c can be coated with reflective material to reflect the excitation light back towards the waveguide to increase the power density of excitation light impinging on the emission detection reagent for the purpose of increase the emission signal.

The waveguides are elongated objects, with a long dimension and shorter cross-sectional dimension. The analyte capture surfaces of the waveguide can be part of the mixing flow chamber wall, but typically they are within the mixing flow chamber but not part of the wall. The waveguides can be oriented along the long axis of the mixing flow chamber or along the short axis of the mixing flow chamber. The flow of the sample can be along the long dimension of the waveguide or perpendicular to the long dimension of the waveguide.

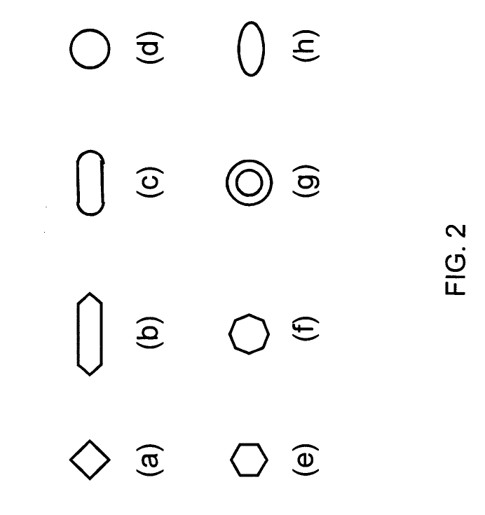

In the embodiment depicted in FIG. 1c, the waveguide member 101 has a rectangular shape. Alternative geometries of waveguide 101 can also be used to carry out the object of the invention. Referring to FIGS. 2a-2h, a non-exhaustive list of several waveguide geometries is presented. As shown in these Figures, a cross-section of waveguide member 101 can have a circular shape, square shape, a ring shape, a polygonal shape (for example, rectangular, trapezoidal, hexagonal or octagonal shape), an annular shape, an oval shape, or any combination or permutation of these and any other useful shape that can guide electromagnetic radiation. In this invention, the waveguide can have any cross-sectional solid or hollow shapes that have low propagation loss in the long direction. In other words, the present invention is not limited to a particular waveguide shape.

The optically clear top of the mixing flow chamber wall 220 and/or the optically clear bottom of the mixing flow chamber wall 230 can also act as waveguides, with the analyte capture surface on the waveguide. In this invention, the waveguide can be made with any material transparent to the excitation light and into which the light emitted by the emission detection reagent can be guided. Typically, waveguide member 101 in one embodiment of the invention can be made of, but not limited to, glass, polymers, optical epoxies, quartz, polypropylene, polyolefin, polystyrene, etc.

The waveguide length can be same as, shorter than, or longer than the mixing flow chamber. It can extend outside the mixing flow chamber on one end or on both ends.

As is known in the art, the ability of a waveguide to confine and direct the propagation of light is dependent on the index of refraction of the waveguide material as well as the index of refraction of material in close proximity. The higher the index of refraction of the waveguide material, compared to its surroundings, the better the waveguide can confine the light for identical geometries. Therefore, it is preferable that the refractive index of waveguide member 101 have a value greater than the refractive index of the medium surrounding the waveguide inside the mixing flow chamber.

The waveguide surface can be multi-layered. In fiber optics, for example, a thin layer of cladding, a material with an index of refraction less than that of the core material, is used to better confine the emitted radiation within the fiber. This same principle can be applied to the waveguide. All or parts of the waveguide can consist of a core surrounded by a cladding. In the embodiment described in FIGS. 1a, 1b & 1c, as an example, all of the parts of the waveguide member 101 except some portions in the interior of the mixing flow chamber 240 are provided with, but not required to have, a cladding. The cladding is generally made of glass or plastic. The cladding performs the following functions: reduces loss of light from the core into the surrounding, reduces scattering loss at the surface of the core, protects the fiber from physical damage and absorbing surface contaminants, and adds mechanical strength.

Part of the cladding can be covered with a coating, or “jacket”. The coating is more desirable outside the mixing flow chamber. The coating serves to physically protect the waveguide member from the outside materials and to prevent any parasitic or environmental radiation from entering into the waveguide.

Some of the waveguide can have a portion outside the mixing flow chamber, part 104, or none at all extending outside the mixing flow chamber.

Parts of the waveguide can be covered with reflective material. In the embodiment described in FIG. 1b, a reflective member 215 can be provided at the first end of the waveguide 102, but this is not required. This member reflects light towards the direction of the detector end of the waveguide 103. Preferably, reflective member 215 is composed of a coating of material that specifically reflects the radiation emitted by the label. It can also be desired that this coating of material absorbs the excitation light in order to limit background radiation reaching the detector. Reflective member 215, which is secured at the first end 102, can also be affixed at first end 233 of mixing flow chamber 240, as is represented in the embodiment of FIG. 1b.

It is not desirable to have the left side 105 and right side 106 of waveguide 101 as shown in FIG. 1c to be analyte capture surface, because there would not be adequate amount of excitation light impinging on side 105 and 106 to impinge on the light detection label. Furthermore, covering the sides 105 and 106 with reflective material, cladding material or other materials different from the waveguide are methods to accomplish this goal and improve transmission of the emission signal to the detector.

The waveguide end 102 can be used to manipulate the reflection of total light power and also to manipulate the reflection of light as a function of wavelength. The use of reflective material is one method of obtaining nearly total reflection for a range of wavelengths of labels. Semi-circular shaped ends can also provide good reflection of light. To obtain frequency selective reflections, multi-layer coatings or gratings can be used, for example, to obtain high reflection of light produced by the labels and low reflection of excitation light. For example, an optical grating is fabricated on the inside of an optical fiber. The grating end of the optical fiber is placed just before the detector. The signal collected by the fiber constitute of both the emission and excitation light. The grating will provide high transmission of the light produced by the labels and low transmission of excitation light to the detector. Thus, the signal to noise ratio of some detectors can be improved.

The number of waveguides inside a mixing flow chamber can be more than one. The arrangement of the waveguides inside the mixing flow chamber can vary.

The inlet 260 and outlet 261 are located on the mixing flow chamber surface 230 opposite that of the radiation transmissive surface 220 in FIG. 1b, but it could be on any part of the boundaries of the mixing flow chamber 240, including sides 231, 232, 233 and 234, or top 220. There can be more than one inlet and more than one outlet. The inlets and the outlets do not have to be on the same wall. The inlets and outlet can have different dimensions and any construction.

FIG. 1a shows one detector system at the end of waveguide end 103. Another detector system can also be implemented at the waveguide end 102, instead of a reflective mirror.

The mixing flow cartridge can contain not only the mixing flow chamber and the waveguide, but can also contain a sample chamber, and chambers that store reagents needed for the assay and waste products from the assay and other preparatory processes.

Signal detection is performed with a detector member 270 provided at one or both extremities of waveguide member 103. The detector member 270 is part of the sensor instrument. Generally, detection can be performed without the presence of fluid inside the chamber. Yet, it should be kept in mind that detection in the present invention can also be done with a fluid continually flowing through the mixing flow chamber. For instance, detection can be done with a reagent or a rinse present inside the mixing flow chamber. Detector member 270 is constructed and arranged to receive a signal exiting second end 103 and to provide quantitative and qualitative information about the assayed sample.

As mentioned previously, the sensing system 200 can be embedded in a sensor instrument. This instrument should be designed to facilitate the mixing flow cartridge installation, radiation illumination and detection. The instrument includes all of the elements necessary to perform detection and analysis in any type of environment. The instrument can also include other functions.

This instrument can be used in the following way for one type of sandwich immunoassay, an example of which is described in this paragraph. First, the sample containing the analyte is introduced into the mixing flow cartridge. A system of filters interposed before the inlet can be used to prevent large particles from entering and clogging the mixing flow chamber. In this mode of operation, the analyte capture surface specific to the analytes has been coated in advance on the waveguide member. The sample containing the analyte is flowed inside the mixing flow chamber between the inlet and the outlet. Analyte that is specific to the capture antibody binds to the waveguide member via the capture antibody, while other matter present in the solution is flushed out of the mixing flow chamber. A rinse can be provided in order to eliminate unbound analyte and any matter that has been partially or non-specifically bound to the waveguide or other surfaces in the mixing flow chamber. For sandwich assay, the emission detection reagent can next be introduced and to bind to the analyte of the analyte/capture antibody complexes, thereby completing the sandwich assay. A further rinse step can be performed to eliminate unbound emission detection reagent. Then, the waveguide is illuminated by a light source. The illumination can take place while rinsing solution is still inside the mixing flow chamber or while the mixing flow chamber is empty. Finally, the signal produced by the emission detection reagent is captured by the waveguide member and guided to the radiation detection member.

This instrument can be used in another way for a second types of sandwich immunoassay, an example of which is described in this paragraph. The waveguide is coated with avidin. First, one or more filters are used to extract large debris from the sample containing the analyte. This is followed by mixed the sample with emission detection reagent and the analyte recognition coating. The analyte recognition coating can be biotinylated antibody. The analyte of interest will be coated with both the analyte recognition coating and the emission detection reagent. The unbound analyte recognition coating and the emission detection reagent can be filtered out and the analyte along with other particulars will be washed and resuspended in buffer. The resuspended solution is flowed inside the mixing flow chamber between the inlet and the outlet. Analyte that is specific to the analyte recognition coating binds to the analyte recognition surface on the waveguide member via the avidin-biotin binding, while other matter present in the solution is flushed out of the mixing flow chamber. A rinse can be provided in order to eliminate unbound analyte and any matter that has been partially or non-specifically bound to the waveguide or other surfaces in the mixing flow chamber. The waveguide is illuminated and the signal produced by the labels on the surface of the waveguide is captured by the waveguide member and guided to the radiation detection member.

This instrument can also be used in other types of sandwich assays.

There are many other types of assays. This sensor is not limited to the sandwich immunoassay.

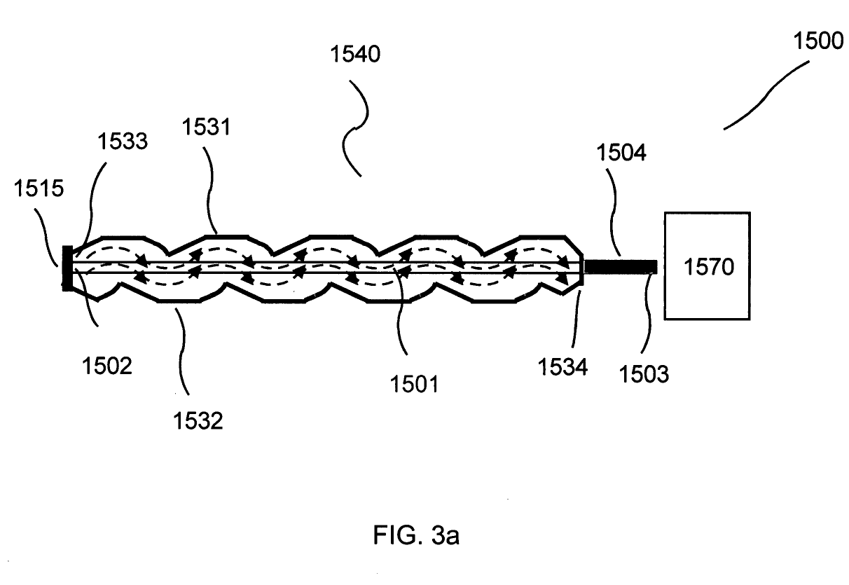

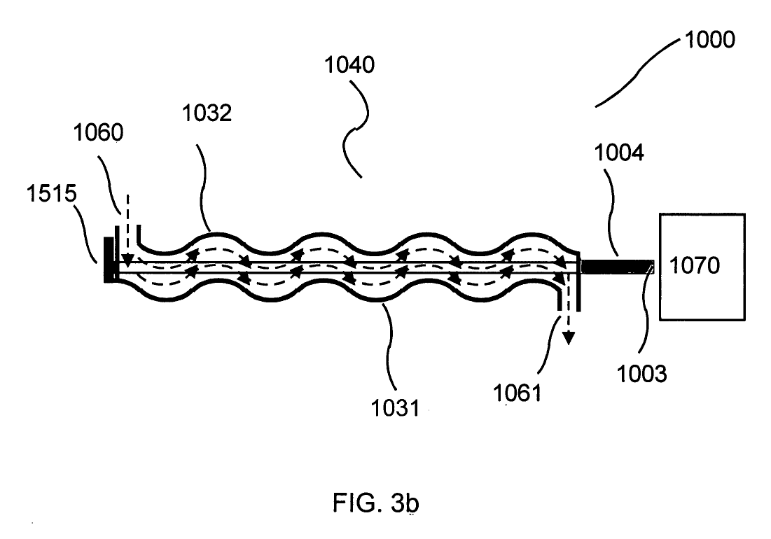

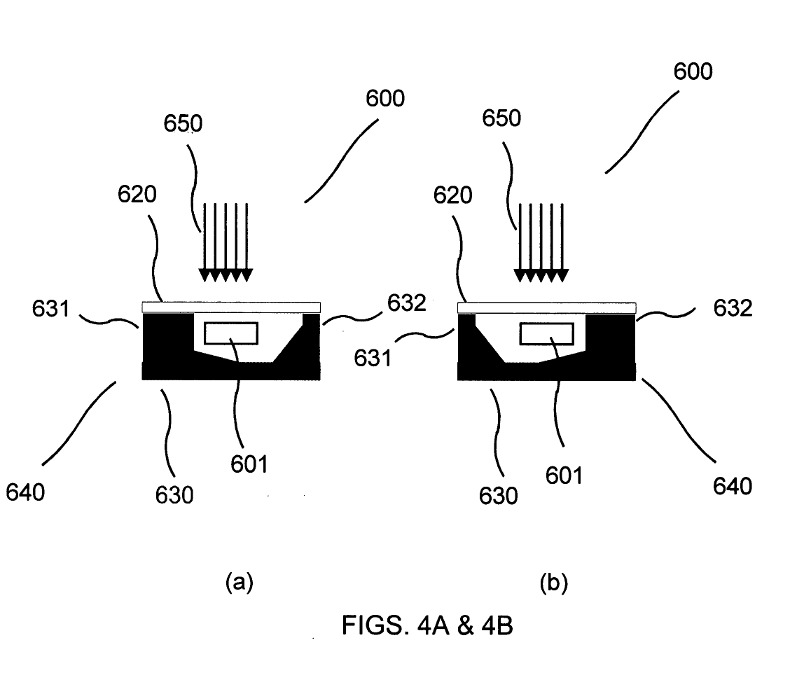

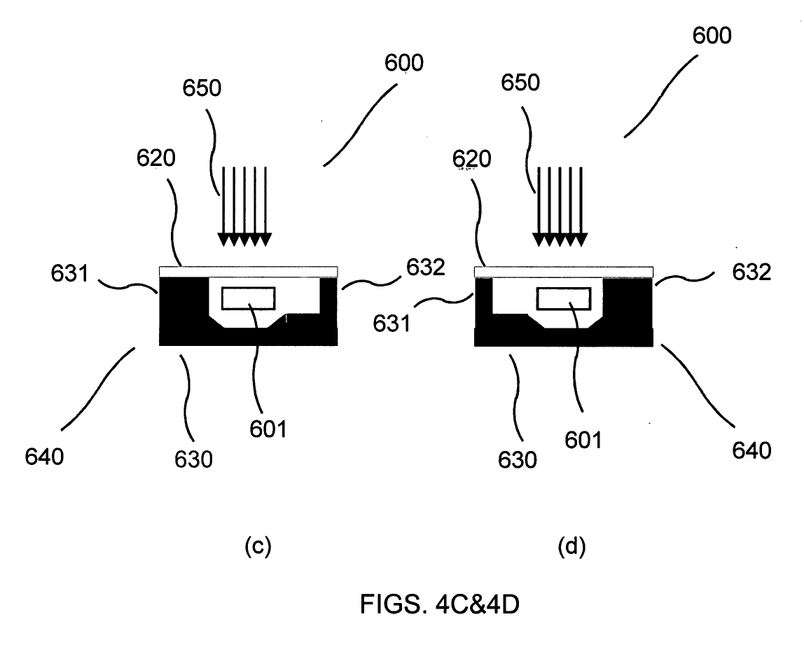

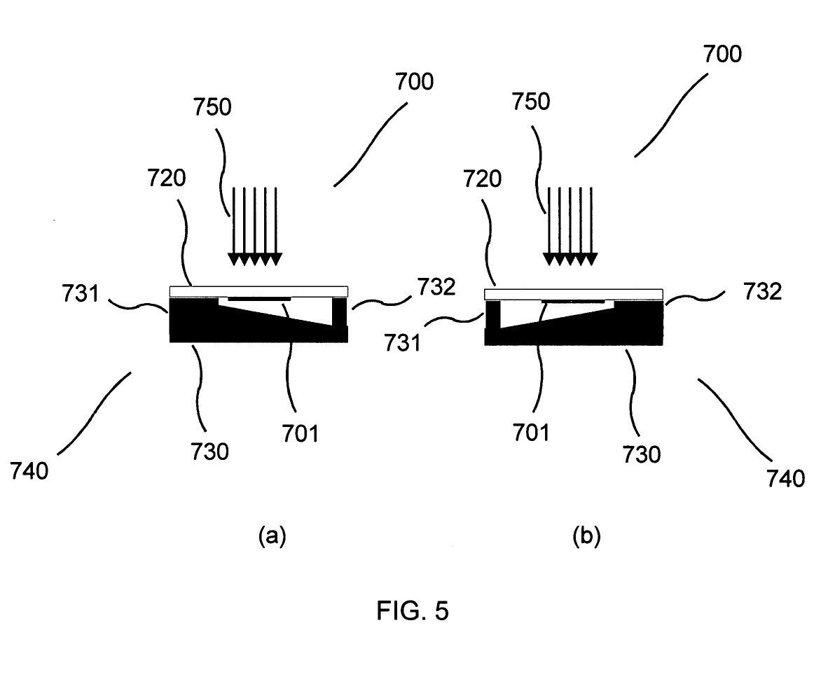

As mentioned in the foregoing discussion, the surface of the mixing flow chamber can have a shape that differs from the one represented in FIGS. 1a, 1b and 1c. Referring to FIGS. 3a and 3b, two alternative examples of side view shapes are provided. Referring to FIGS. 4a, 4b, 5a and 5b, alternative examples of end view surface shapes are provided. The embodiment of the possible shapes of the mixing flow chamber is not limited to these examples.

As shown in FIGS. 3a and 3b, the side views of the mixing flow chambers 1540 can have different undulating forms.

Three-dimensional mixing flow surface can also be used in another embodiment of the invention. One such embodiment is represented in FIGS. 4a and 4b at two axial locations, and another such embodiment is represented in FIGS. 4c and 4d at two axial locations representing cross-sectional end-views of a mixing flow-through sensor according to different embodiments of the invention. Similar to the embodiment depicted in FIG. 1 c, the sensing system 600 comprises waveguide member 601 that is located inside the elongated body of mixing flow chamber 640. Elongated body 640 includes top transmissive portion 620, which is transparent to the beam of radiation 650 impinging on waveguide member 601. Elongated body 640 further includes side member 631 and 632 and a bottom member 630. Like the embodiment shown in FIG. 1c, boundary members 630, 631 and 632 can be made capable of absorbing the excitation light and therefore reduce the scattering of light towards the detector member. FIGS. 4a-b and 4c-d illustrate the mixing flow chamber 640 constructed such that (a) at certain positions the left wall 631 is closer to the waveguide and (b) at other positions the right wall 632 is closer to the waveguide, respectively. FIGS. 4a-b and 4c-d not only show that the side walls are undulated, but that the bottom wall 630 also undulates in the long direction.

FIGS. 5a and 5b present cross-sectional end-views at two axial locations of another embodiment of the mixing flow-through sensor where there is no waveguide in the interior of mixing flow chamber 740. Similar to the embodiment depicted in FIG. 1c, the sensing system 700 consists of an elongated body 740 and transmissive top member 720. A portion of the transmissive top member 720 is coated with the analyte capture surface 701 and the transmissive top member 720 also serves as the waveguide. Elongated body 740 further includes side member 731 and 732 and a bottom member 730. Like the embodiment shown in FIG. 1c, boundary members 730, 731 and 732 are capable of absorbing the radiation emitted by the light source and can therefore reduce the scattering of light towards the detector member. FIGS. 5a and 5b illustrate the mixing flow chamber 740 at the positions (a) where the members 730 and 731 are closer to the waveguide and (b) where the members 730 and 732 are closer to the waveguide, respectively.

Although several illustrations of mixing flow surfaces have been provided in the foregoing discussion, it should be understood that other mixing flow members or mixing flow surfaces can be suitable to carry out the object of the invention.

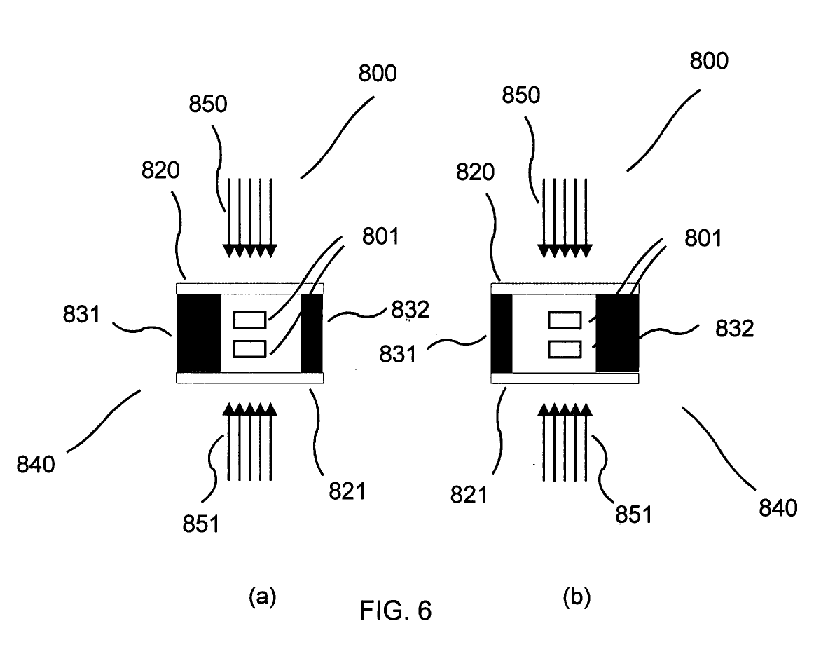

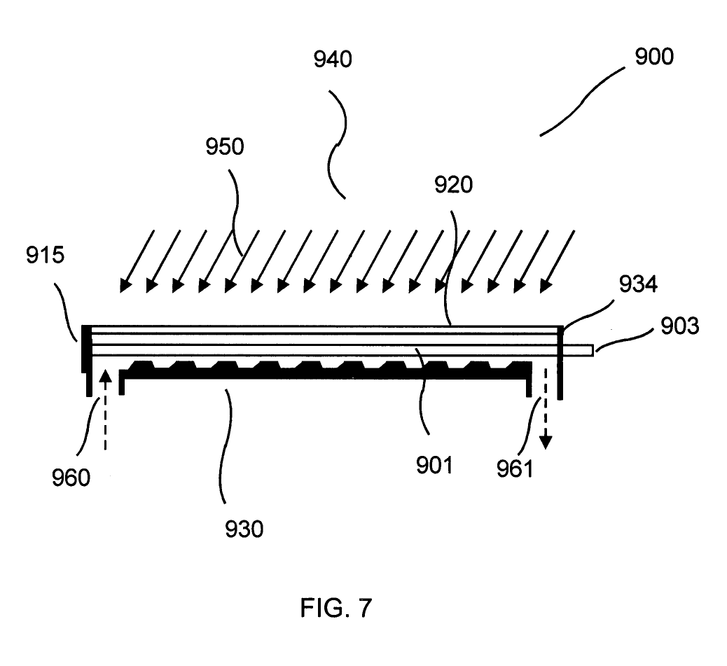

In order to increase the emission signal received by the detector member, the mixing flow chamber can optionally contain more than one waveguide. Such embodiment is represented in FIGS. 6a and 6b presenting two cross-sectional end-views of a mixing flow-through sensor at two different axial locations. The sensing system 800 consists of two waveguide members 801 that are located inside elongated body of mixing flow chamber 840. Elongated body 840 includes top transmissive member 820 and bottom transmissive member 821, which are transparent to the excitation light 850 and 851 impinging on waveguide members 801. Like the embodiment shown in FIG. 1c, boundary walls 831 and 832 can be made capable of absorbing the excitation light. FIGS. 6a and 6b illustrating the mixing flow chamber 840 at the positions that (a) the left wall 831 is closer to the waveguide and (b) the right wall 831 is closer to the waveguide, respectively.

FIG. 7 presents side view of a mixing flow-through sensor according to another embodiment of the invention at two different axial locations. The sensing system 900 comprises waveguide members 901 that are disposed inside elongated body of mixing flow chamber 940. The end of the waveguide 903 is unobscured by the waveguide wall 934 to let the emission light out to the detector, but not extended outside the wall 934. Elongated body 940 includes top transmissive member 920 and bottom light absorbing member 930 and an inlet 960 and outlet 961. The bottom wall member 930 is undulating. The excitation light 950 is collimated but not perpendicular to the long direction of the waveguide.

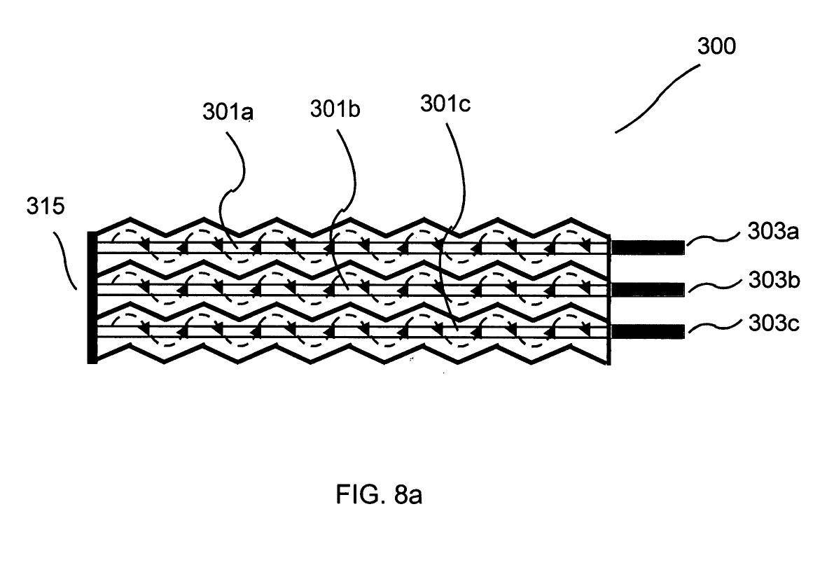

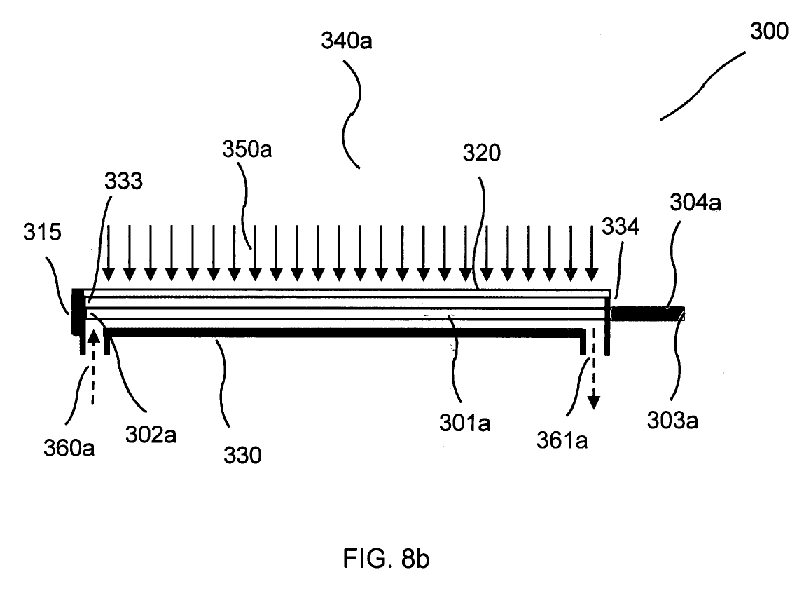

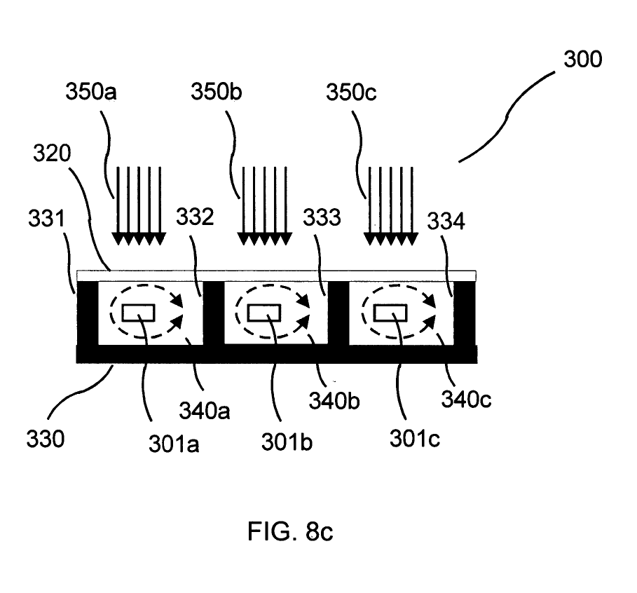

FIGS. 8a, 8b and 8c depicts a top view, side view and an end view of the multi-analyte sensor according to one embodiment of the invention. Multi-analyte sensor 300 comprises a plurality of mixing flow chambers, each of them housing a waveguide member capable of conveying the emitted light to a detector member (not shown in FIG. 8a). The multi-analyte sensor 300 is constructed and arranged to identify and quantify different analytes at the same time or at different times. It also allows for an optimal construction of the sandwich assays on each of the waveguides due to the mixing flow chamber.

Multi-analyte sensor 300 comprises a plurality of mixing flow chambers 340a, 340b and 340c grouply secured. Like the embodiment depicted in FIG. 1a, each of the mixing flow chambers 340a, 340b and 340c respectively comprise a waveguide member 301a, 301b and 301c on which is the analyte capture surface so as to substantially expose the entire analyte capture surface of the waveguide to the sample in the interior of the mixing flow chamber. The first end of each waveguide can be coated by a reflective or multi-layered material or shaped to improve the reflection of emitted radiation and reduce the reflection of the emitted radiation. The second end of each waveguide can be unobscured to allow the transmission of the emitted radiation out of the waveguide to the detector system (not shown).

FIG. 8b shows the side view of the mixing flow chamber 340, which is further comprised of radiation transmissive surface 320 allowing the excitation light to propagate to the analyte capture surface of the waveguide. The lower border 330 can be clear, black or any other color, or coated with reflective or absorbent material. The mixing flow chamber includes inlets 360a-c and outlets 361a-c to allow a fluid to flow inside the mixing flow chamber between inlet 360a-c and outlets 361a-c, respectively. The excitation light 350 impinges directly or indirectly on the waveguide surface.

FIG. 8c shows the end view of the sensing system 300, including radiation transparent top boundary 320, the side members 331, 332, 333 and 334, the bottom wall 330, the waveguides 301a, 301b and 301c, and incident radiation 350.

Mixing flow surfaces are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

While the number of mixing flow chambers is limited to three in the embodiment of FIGS. 8a, 8b and 8c, it should be apparent to one skilled in the art to which the invention pertains that multi-analyte sensor can comprise a larger number of mixing flow chambers. Generally, the number of mixing flow chambers depends on the application needs and can be determined by the size of the instrument.

All the variations of inventions described earlier for FIGS. 1a, 1b and 1c are also applicable to this multi-mixing flow chambers sensor embodiment, FIGS. 8a, 8b and 8c.

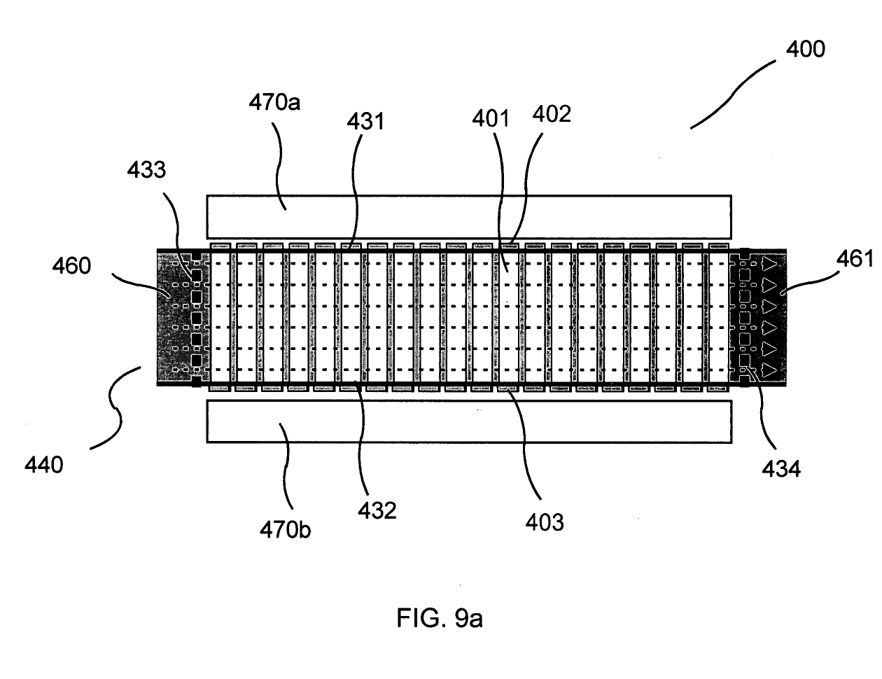

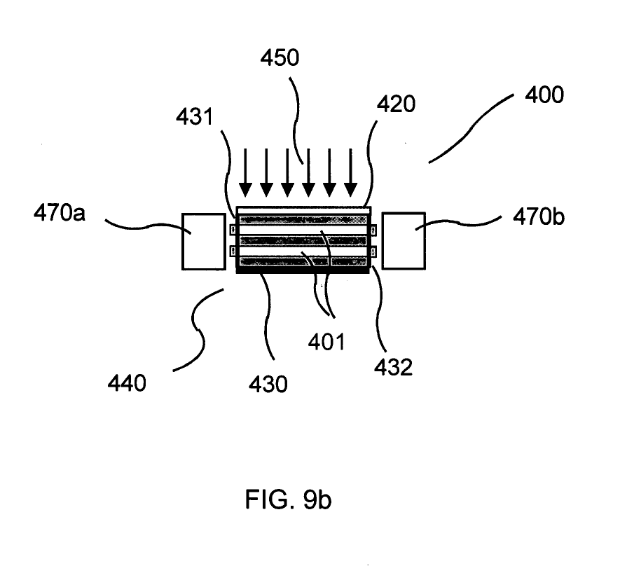

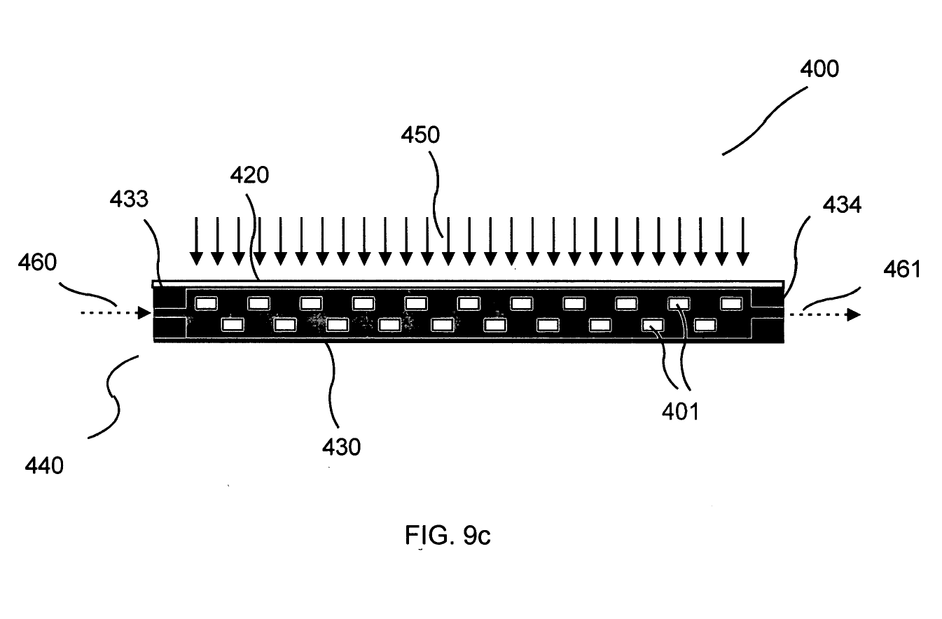

FIGS. 9a, 9b and 9c show the top view, side view and end view, respectively, of a mixing flow-through sensor providing fast flow rate and rapid capture of the analyte according to another embodiment of the invention. Sensing system 400 is comprised of a mixing flow chamber 440, with a large number of waveguide members 401 coated with analyte capture surface, and the detector system members 470a and 470b.

In the embodiment shown in FIG. 9a, waveguide members 401 consist of a number of elongated members, adapted to propagate along their lengths the collected emission signal. Sensor 400 comprises a plurality of waveguide members 401 in the mixing flow chamber 440, so as to expose substantially all of the waveguide surface to the sample, leaving first end 402 and second end 403 of the waveguide unobscured. More particularly, mixing flow chamber 440 is comprised of elongated side bodies 431 and 432 that extend outward from waveguide member 401 and is constructed and arranged to contain a portion of the waveguides. The waveguides 401 are positioned approximately perpendicular to the flow of the sample. The side members 431 and 432 are secured to waveguide members 401. The inlet 460 and outlet 461 allow a fluid sample to flow inside the mixing flow chamber between inlet 460 and outlet 461 and they are formed by holes through mixing flow chamber walls 433 and 434, respectively. Two detector members 470a and 470b can be used to detect light exiting from the waveguide ends 402 and 403.

As the fluid flows through the mixing flow chamber over the waveguides, the analytes in the fluid has improved chance of being captured if the number of waveguides is increased. The waveguides can capture one or more varieties of analytes.

FIG. 9b shows the side view of the mixing flow chamber 440 further comprises of radiation transmissive surface 420 allowing the excitation light to propagate to the analyte capture surface of the waveguide, the side walls 431 and 432, and the lower border 430, which can be clear, be black or be coated with reflective material. The excitation light 450 impinges on the waveguide surfaces. The emission signal exits from the waveguide ends to enter the detector members 470a and 470b.

FIG. 9c shows the end view of the sensing system 400, including radiation transmissive top boundary 420, the bottom wall 430, side walls 433 and 434, inlet 460, outlet 461, the waveguides 401 and incident radiation 450.

The number of waveguides, their position, and length can vary. Only one of the detector systems can be necessary. The inlet 460 and outlet 461 can be located on the bottom wall 430.

All the variations of inventions described earlier for FIGS. 1a, 1b and 1c are also applicable to this multi-mixing flow chambers sensor embodiment, FIGS. 9a, 9b and 9c.

The mixing of the fluid is caused by waveguides because they are positioned in the path of the fluid flow. The waveguides are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

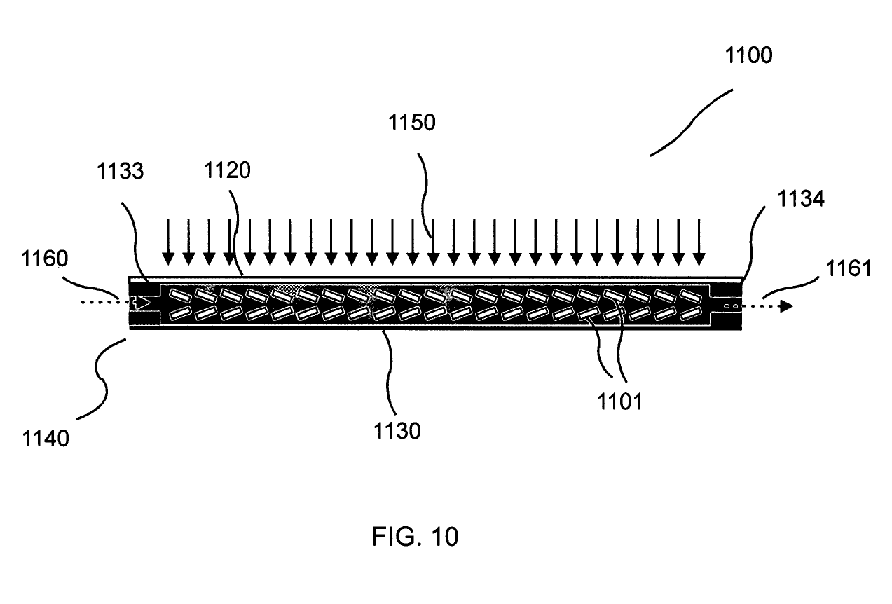

FIG. 10 shows another embodiment of the end view of the sensing system 400. The waveguides are positioned to allow a different flow. FIG. 10 shows the end view of the sensing system 1100, including radiation transmissive top boundary 1120, the bottom wall 1130, side walls 1133 and 1134, inlet 1160, outlet 1161, the waveguides 1101 and incident radiation 1150. When this embodiment is coupled with pulsed, reversible flow direction, the mixing flow chamber 1140 can also provide efficient fluid sampling by the waveguide analyte capture surface.

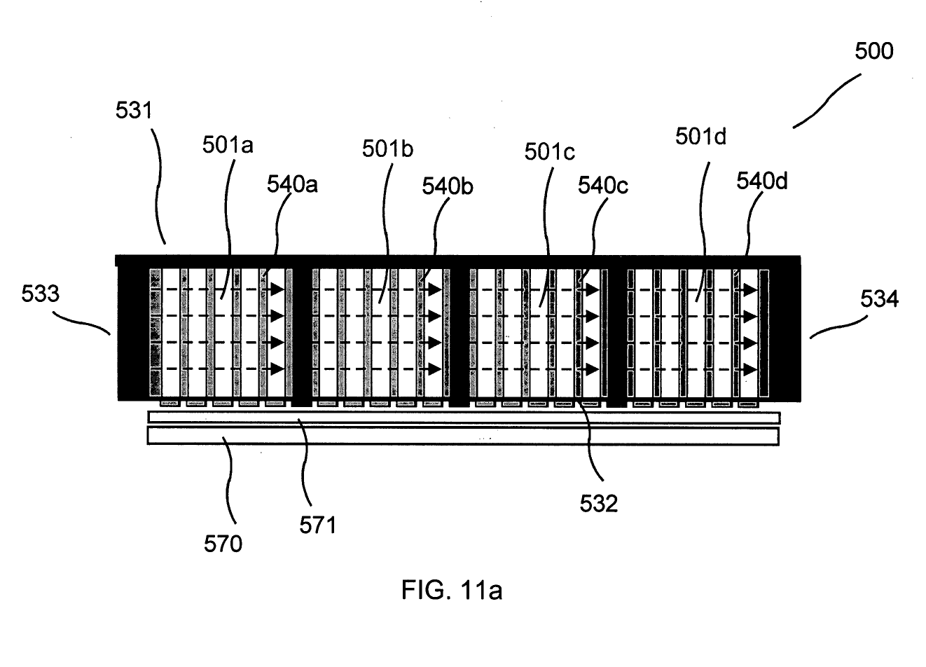

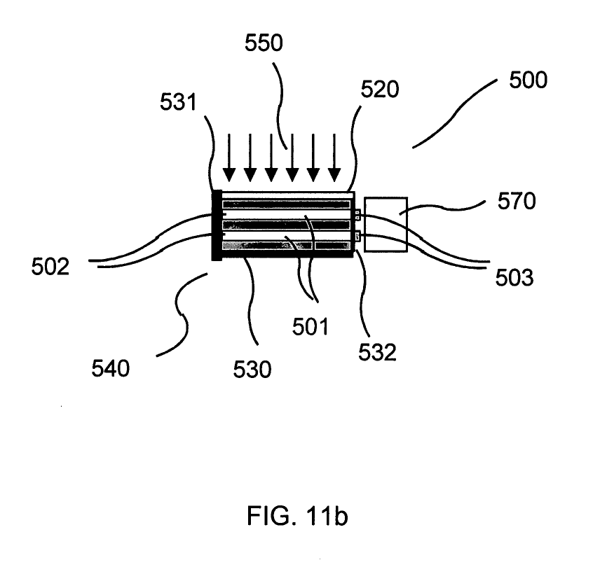

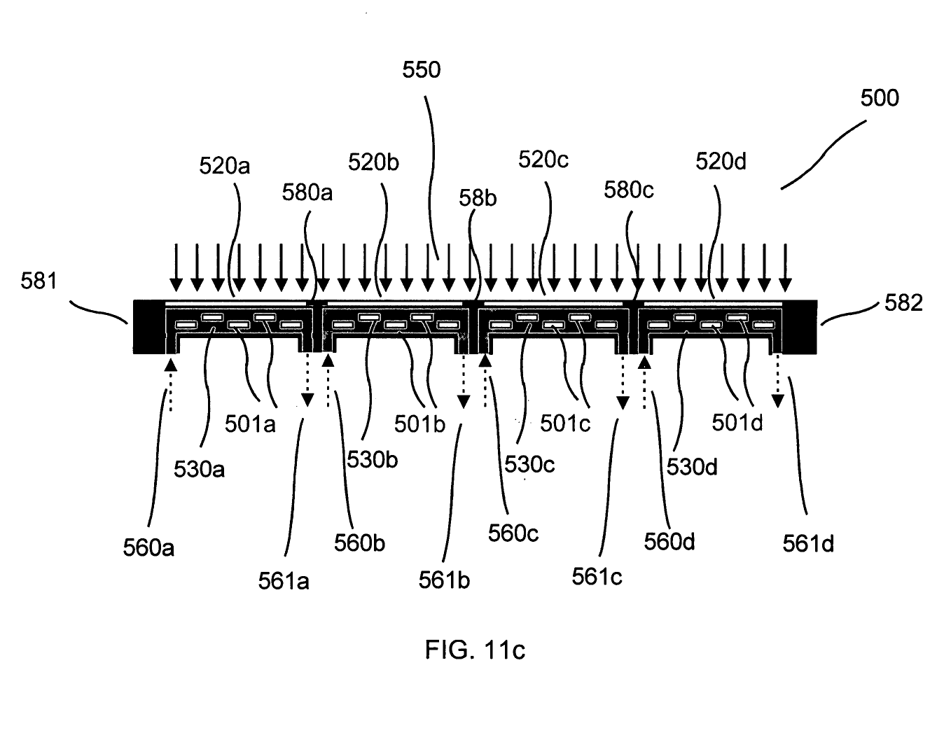

FIGS. 11a, 11b and 11c show the top view, side view and end view of a multi-analyte mixing flow-through sensor according to another embodiment of the invention, respectively. This embodiment is applicable for testing large volumes of samples over large number of waveguides to enable more rapid analyte capture. Sensing system 500 comprises mixing flow chambers 540a, 540b, 540c and 540d, with a large number of waveguide members 501a, 501b, 501c and 501d, coated with analyte capture surface, and the detector systems member 570.

In the embodiment shown in FIG. 11a, the sensing system 500 consists of a number of mixing flow chambers 540a, 540b, 540c and 540d. The waveguide members 501a, 501b, 501c and 501d are situated in the mixing flow chambers 540a, 540b, 540c and 540d, so as to expose substantially all of the waveguide surface to the sample, leaving first end 502 and second end 503 of the waveguide unobscured. The waveguides 501a, 501b, 501c and 501d are positioned approximately perpendicular to the flow of the sample. The waveguide members 501a, 501b, 501c and 501d are secured to the side members 531 and 532. The emission signal exits from the waveguides and enters the detector member 570.

FIG. 11b shows the side view of the mixing flow chamber 540 further comprises of radiation transmissive surface 520 allowing the excitation light to propagate to the analyte capture surface of the waveguide, the side walls 531 and 532, and the lower border 530, which can be clear, black or any other color, or coated with reflective or absorbent material. The excitation light 550 directly incident on the waveguide surfaces. The waveguide end 502 can be coated with a reflective material. The emission signal exits from the waveguide end 503 to enter the detector system 570.

FIG. 11c shows the end view of the sensing system 500, including radiation transmissive top boundary 520, the bottom wall 530, side walls 531 and 532, the waveguides 501 and incident radiation 550. The fluid enters each mixing flow chamber 540a, 540b, 540c and 540d through inlets 560a, 560b, 560c and 560d and exit through outlets 561a,561b, 561c, and 561d, respectively.

The mixing of the fluid is caused by waveguides because they are positioned in the path of the fluid flow. The waveguides are constructed and arranged to maximize the interaction between the constitutive elements of the fluid solution and the waveguide member. Specifically, mixing flow surface is constructed and arranged so that the fluid flowing inside each mixing flow chamber is in a non-laminar regime.

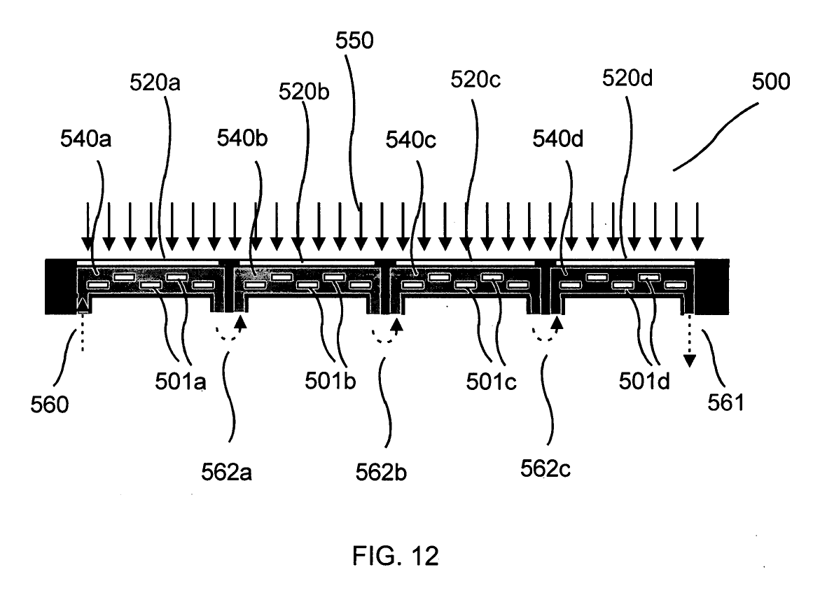

FIG. 12 shows the flow of the sample over all waveguides is achieved by sending the sample from outlets from one mixing flow chamber to the inlet of the next mixing flow chamber during the analyte capture phase. During the rest of the procedures, the solutions from one mixing flow chamber preferably do not go to the next mixing flow chamber.

The number of waveguides, their position and lengths can vary. Detector systems can be used on either or both ends of the waveguides. The inlet 560 and outlet 561 can be located on the bottom wall 530.

All the variations of inventions described earlier for FIGS. 1a, 1b and 1c, FIGS. 11a, 11b and 11c are also applicable to this multi-mixing flow chamber sensor embodiment,

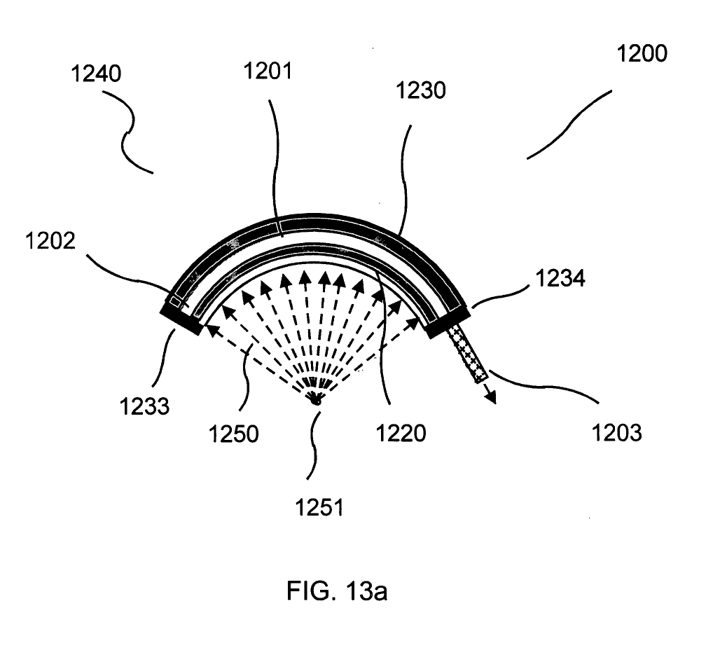

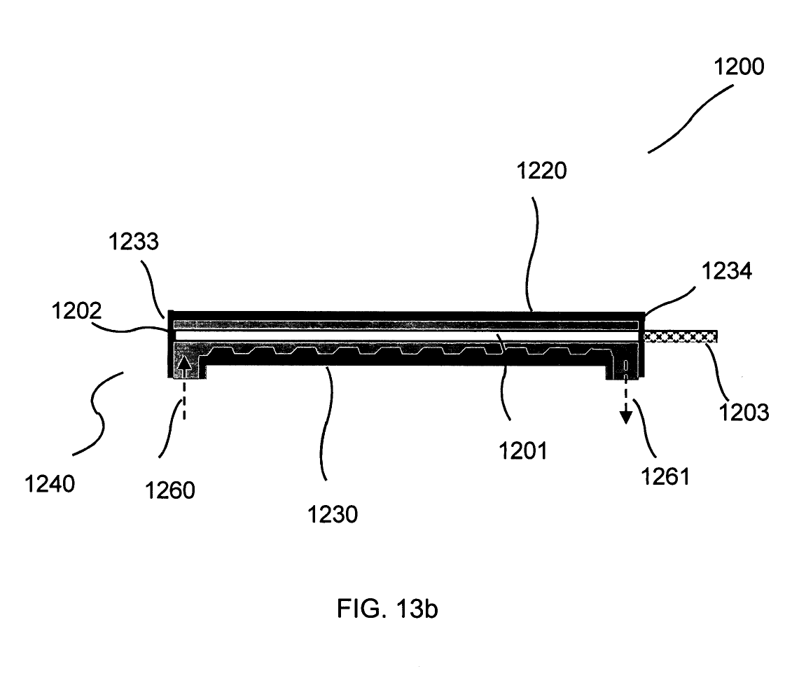

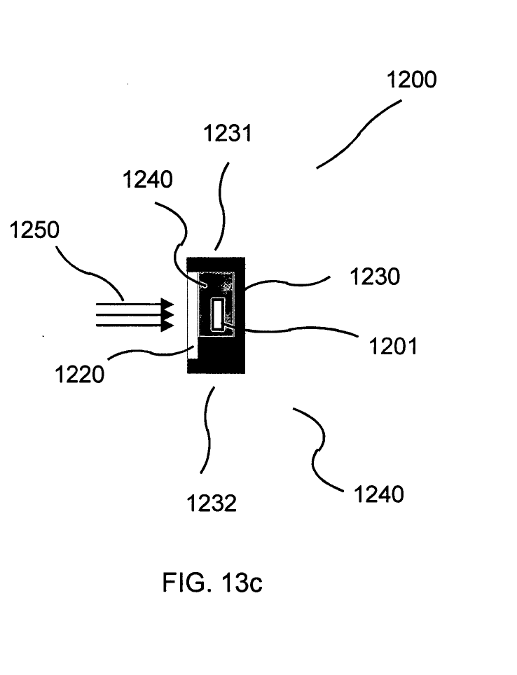

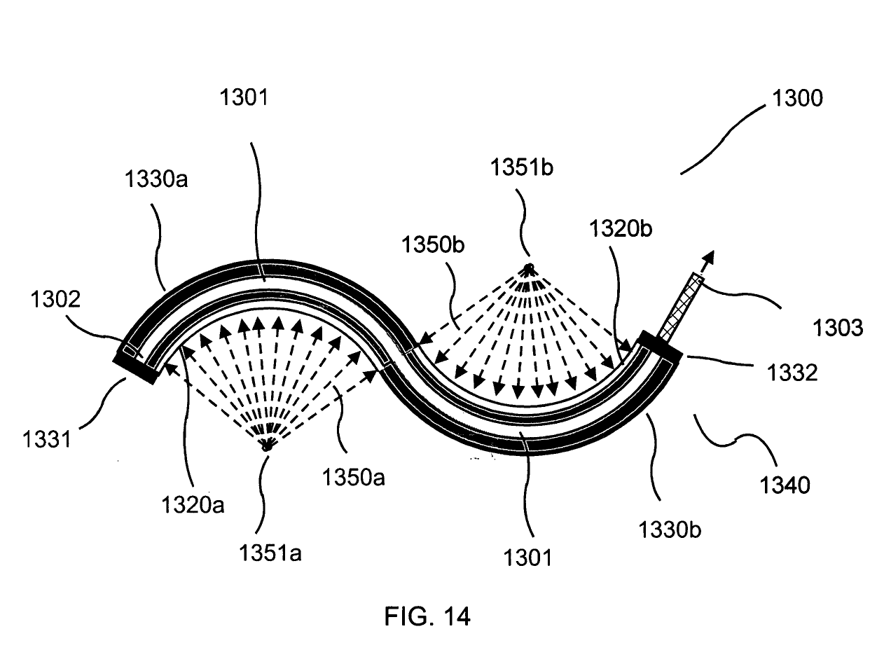

The sensor can also be achieved with an embodiment utilizing a diverging light source as shown in the cross sectional top view, side view and end view represented in Figure 13a, 13b and 13c, respectively. It provides a perpendicular irradiation without using an optical system, thereby reducing the size and optics associated with the system.

In this embodiment shown in FIG. 13a, the mixing flow chamber 1240 is bent in the form of a section of a circle. The mixing flow chamber 1240 is comprised of elongated side bodies 1220 and 1230, and end bodies 1233 and 1234. The waveguide 1201 is secured to the end members 1233 and 1234.

Light from a point source 1251 diverges in a fan beam 1250 and impinges onto radiation transmissive surface 1220 of mixing flow chamber 1240. The waveguide is a curved elongated member 1201, adapted to propagate along its length the collected emission signal. The waveguide member 1201 passes through mixing flow chamber 1240, so as to expose substantially all of the waveguide surface to the sample, leaving first end 1202 and second end 1203 of the waveguide unobscured. A reflective surface can be placed on the first end of waveguide 1202. The emission signal is transmitted out of the end 1203 into detector member (not shown).

FIG. 13b shows the side view of the mixing flow chamber 1240 seen through the center of the waveguide. The mixing flow chamber 1240 is further comprised of radiation transmissive surface 1220 allowing the excitation light to propagate to the analyte capture surface of the waveguide, the end walls 1233 and 1234, and inlet 1260 and outlet 1261. The undulating border 1230 can be made of light absorbing material. The undulating boarder 1230 provides the mixing as the sample flows from inlet 1260 to outlet 1261.

FIG. 13c shows the cross sectional end view of the sensing system 1200, including radiation transmissive boundary 1220, the undulating boundary 1230, side walls 1231 and 1232, the waveguides 1201 and incident radiation 1250.

An alternative embodiment that provides multi-analyte sensing and fan light beam is provided in FIG. 14, consisting of two consecutive embodiments shown in FIG. 13a. The sensing system 1300 is comprised of mixing flow chamber 1340 doubly bent, such that projection beams 1350a and 1350b perpendicularly impinge onto radiation transmissive surfaces 1320a and 1320b. As can be seen in this embodiment, the irradiation of waveguide 1301 is provided by two diverging light sources 1351a and 1351b, each being disposed towards a circular section of the mixing flow chamber. In this embodiment, each section is illuminated with a cone beam light source and can be used to detect the same analyte or a different analyte. While only two bent sections are provided in FIG. 14, alternative embodiments containing more sections can also be used to carry the object of the invention.

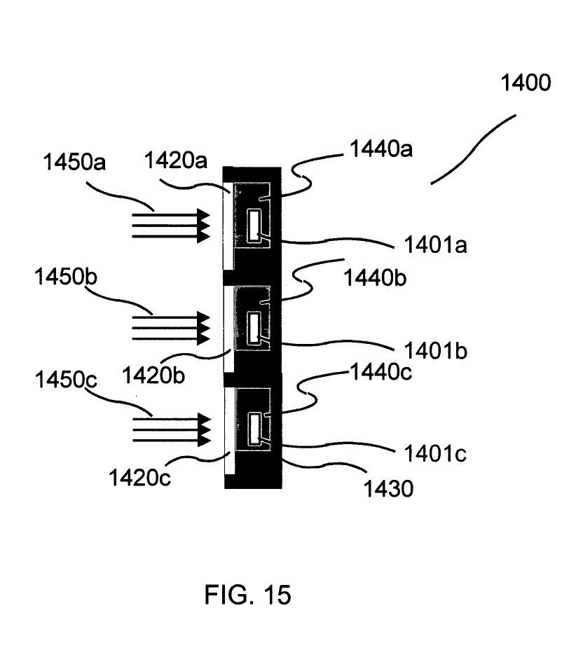

Another alternative embodiment that also provides multi-analyte recognition and fan light beam is provided in the cross sectional end view in FIG. 15. This is applicable to top views shown in FIG. 13a and FIG. 14. The fluid inlet and outlet are to be placed in the wall 1230 in FIG. 13a and wall 1330 in FIG. 14. While the number of mixing flow chambers is limited to three in the embodiment of FIG. 15, it should be apparent to one skilled in the art to which the invention pertains that multi-analyte sensor can comprise a different number of mixing flow chambers. Generally, the number of mixing flow chambers depends on the application needs and can be determined by the size of the instrument.

All the variations of inventions described earlier for FIGS. 1a, 1b and 1c are also applicable to this multi-mixing flow chambers sensor embodiment, FIGS. 13a, 13b, 13c, and 15.

Another alternative embodiment to mix the fluid in the flow chamber is to actuate movable objects in the chamber. For example, the moving objects can be small air bubbles, compressible beads, small magnetic beads or rods. Other means known in the art that facilitate mixing of fluid or fluid-like substances similarly can be used to configure a flow chamber given the teachings and guidance provided herein. The actuation of the objects can be achieved electronically, mechanically, electromechanically, thermally, electromagnetically, magnetically, by vibration or other energy sources. The flow of the sample can be along the length of the waveguide or perpendicular to the length of the waveguide. Two examples among a wide variety of possibilities are given below.

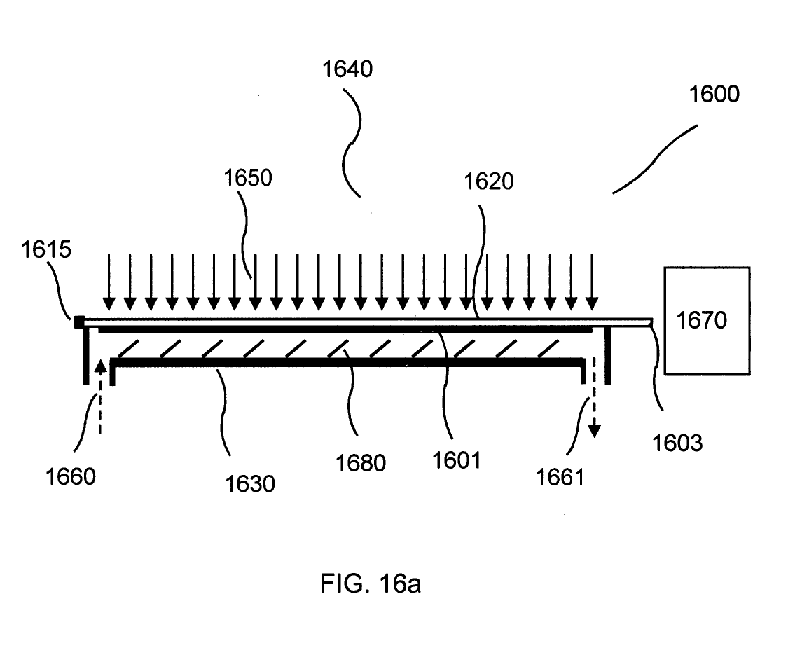

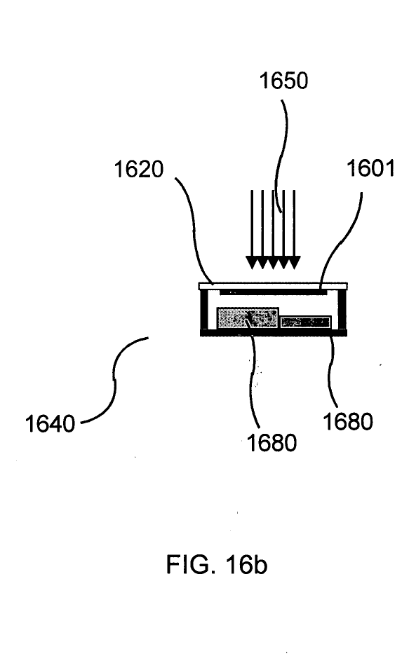

FIGS. 16a and 16b are cross sectional side view and end view of an mixing flow sensor where the flow is along the length of the waveguide and the mixing is achieved by actuation of movable objects 1680 below the waveguide. In this drawing, the waveguide is also the top boundary. The waveguide can also be in the interior of the flow chamber.

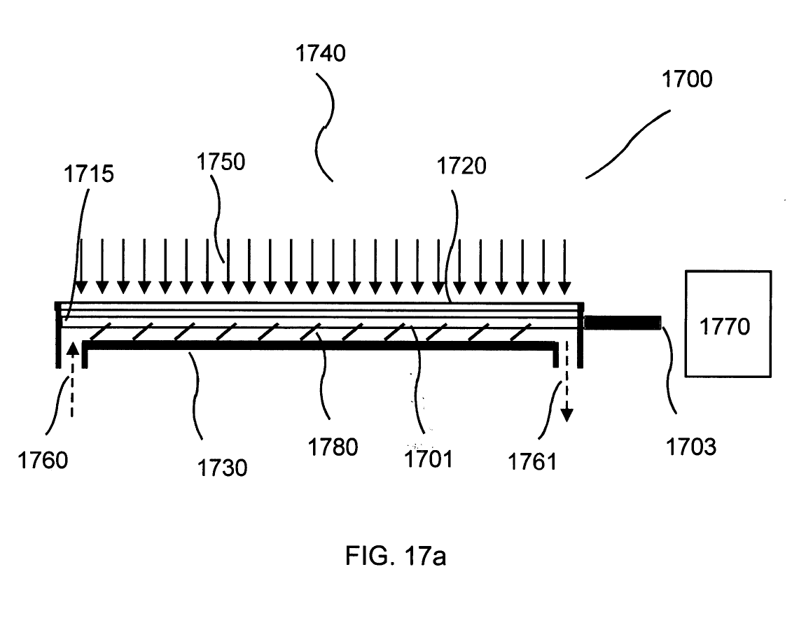

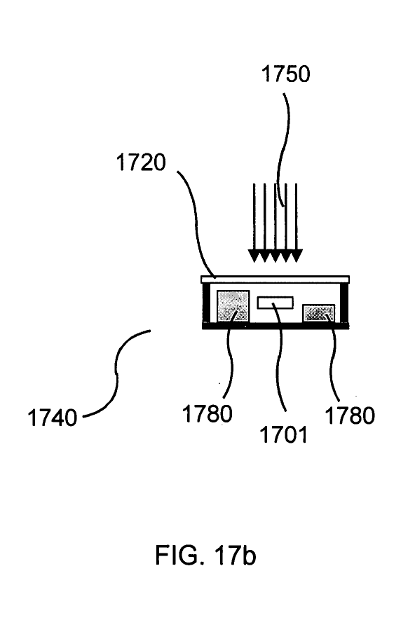

FIGS. 17a and 17b are cross sectional side view and end view of another embodiment where the mixing is achieved by actuation of movable objects 1780 at the sides of the waveguide. The motion of the moving parts on one side can be the same as the moving parts on the other side, but can also be different. The shape of each piece of the moving part can be the same or different. The shape of the moving part can vary and the speed of the motion can also vary temporally.

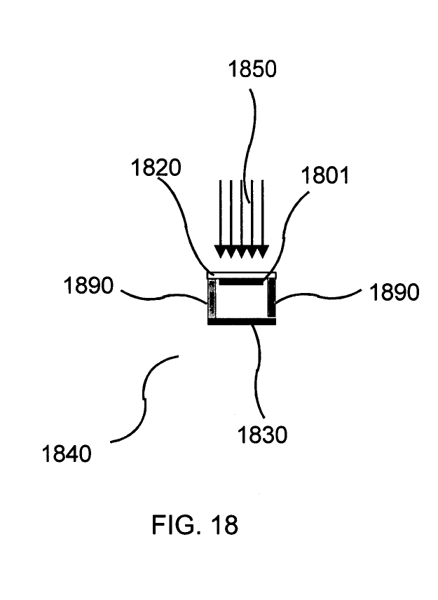

Another alternative embodiment to mix the flow in the flow chamber is to apply an electrical field across the flow chamber in the cross sectional plane. FIG. 18 shows the end view where the application of the electric field is in the vertical direction such that the electric potential on the clear surface 1820 is different from bottom surface 1830 and the side walls 1890 are insulating. Appropriate voltages will be chosen for the analyte to be detected. The flow chamber is not limited to the rectangular shape and the location of the electrodes can vary. The amplitude of the electric field can be uniform or vary in the axial length. The vector of the electric field can also vary in direction along the axial length.

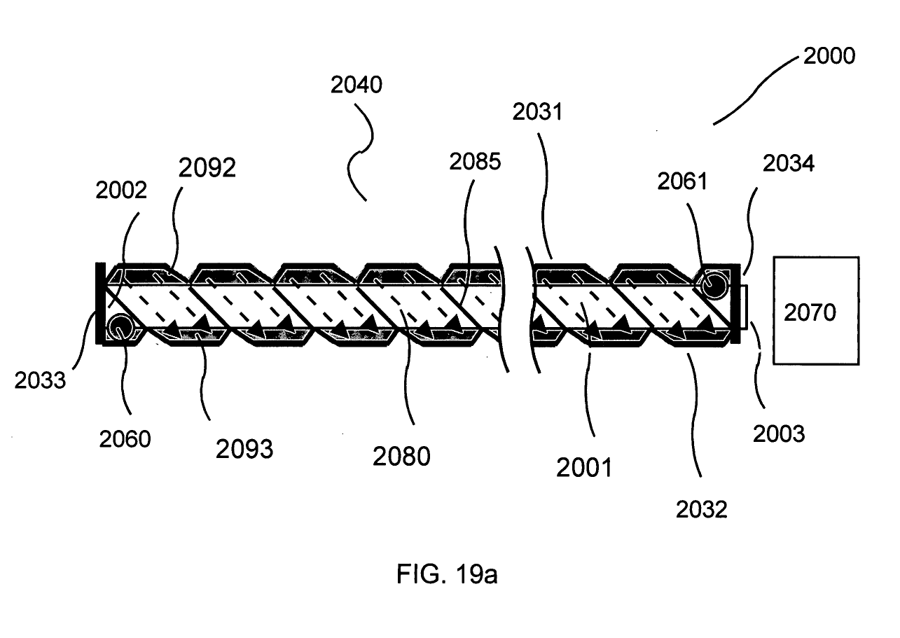

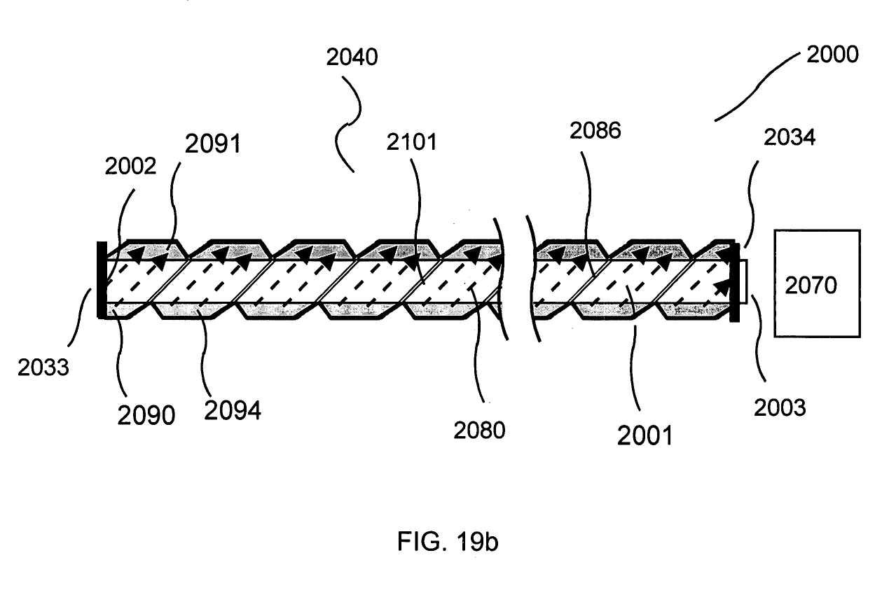

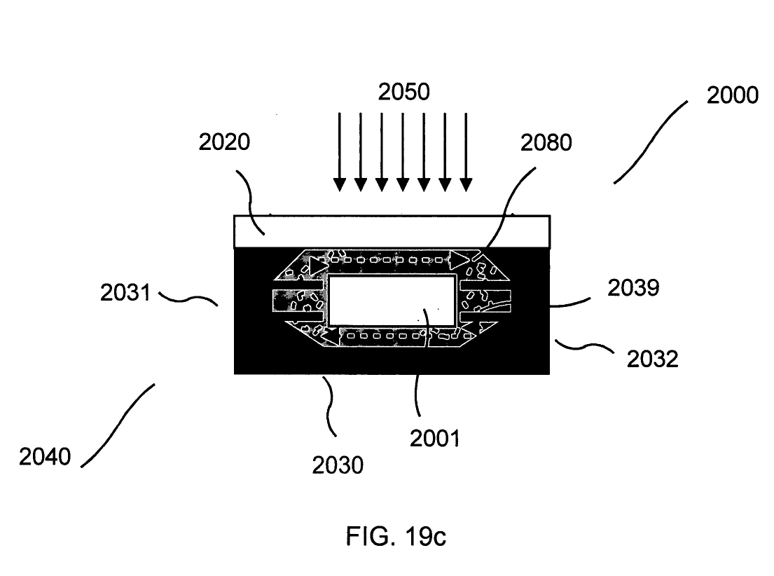

FIGS. 19a, 19b and 19c correspond to a bottom, top and end views of the mixing flow chamber according to one embodiment of the invention where the fluid is guided to flow in a spiral pattern 2080 around the waveguide 2001 and the fluid is mixed at the sides of the waveguide 2031, 2032 and 2039.

FIGS. 19a and 19b correspond to a bottom and top views showing the waveguide 2001, the direction of the flow (dashed arrows) 2080. The sample enters the chamber at the inlet 2060 and exits the chamber at the outlet 2061 at the bottom of the fluidic chip. Fluid flows in a spiral motion as follows: (a) fluid flows from the inlet 2060 to position 2090 over the top of the waveguide to position 2091 and then down to position 2092 at the bottom, and (b) fluid flows from position 2092 under the waveguide to position 2093 and then up to position 2904 at the top. This motion completes one cycle around the waveguide 2001 forming one segment of the flow chamber and the process repeats in additional segments until the end of the waveguide.

FIG. 19c represents an end cross-sectional view showing the waveguide 2001 and the fluid motion 2080 in dashed curves circling around the waveguide in a spiral. The fluid motion is guided by structures above and below the waveguide, not shown here, but is shown in FIGS. 19A and 19B. The fluid is passively mixed on the sides by three-dimensional structures 2039 on the sides of the flow-chamber 2031 and 2032. All the variations of inventions described earlier for FIGS. 1a, 1b and 1c, FIGS. 11a, 11b and 11c are also applicable, for example, to this multi-mixing flow chamber sensor embodiment.

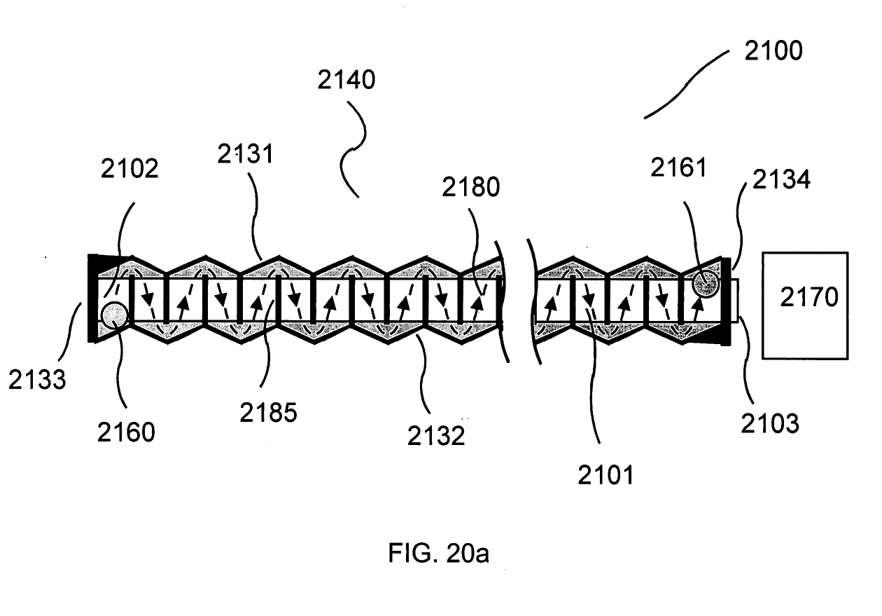

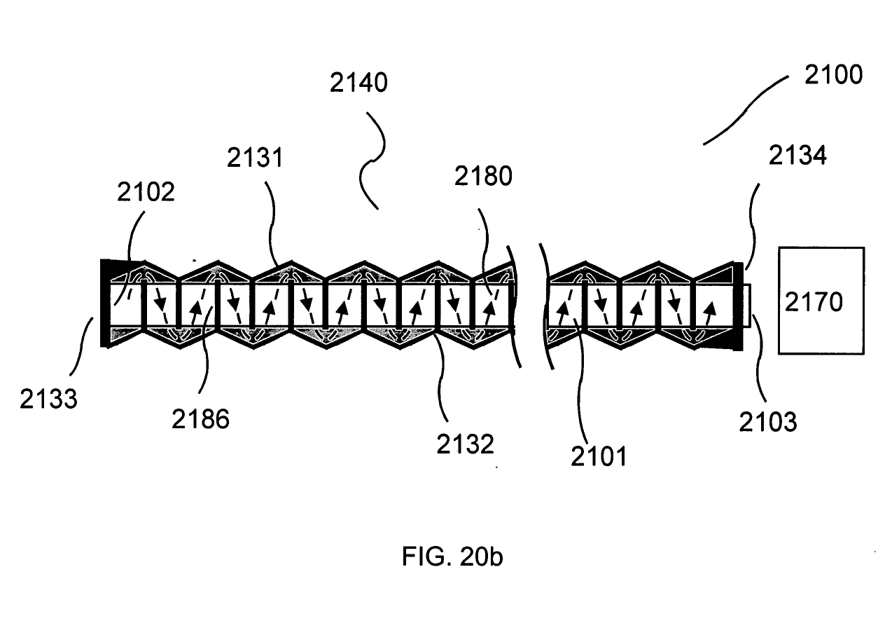

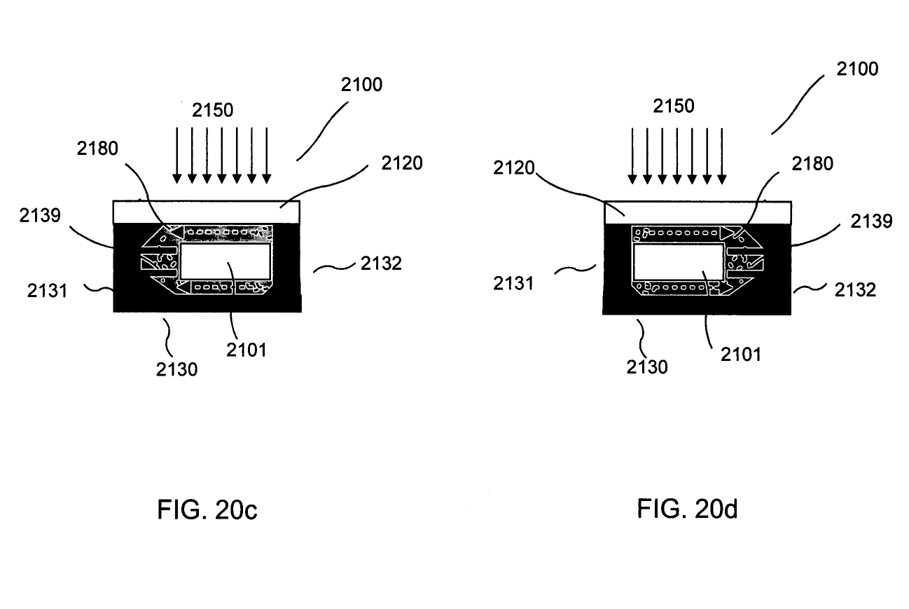

FIGS. 20a, 20b, 20c and 20d correspond to a bottom, top, and end views at one axial location and end view at another axial location of the mixing flow-through sensor according to one embodiment of the invention where the fluid is guided by structures 2185 and 2186 to flow in a zig-zag pattern 2180 across the top and bottom of the waveguide 2101, and the fluid is mixed at the sides of the waveguide 2131, 2132 and 2139.

FIGS. 20c and 20d represent a cross-sectional end view at two different locations showing the waveguide 2101, shape of the flow chamber walls 2139, and the fluid motion in dashed curves 2180. All the variations of inventions described earlier for FIGS. 1a, 1b and 1c, FIGS. 11a, 11b and 11c are also applicable, for example, to this multi-mixing flow chamber sensor embodiment.

Mixing flow waveguide sensor can also be achieved with an embodiment utilizing evanescent wave excitation. The excitation source propagates along the inside of the optical waveguide. The excitation light is not applied from the sides of the waveguide, but input into the waveguide at one end. All the previous description about the wall undulations are applicable to the evanescent wave excitation. In addition, the surfaces 220 in FIGS. 1b and 1c, the surfaces 620 in FIGS. 4a, 4b, 4c and 4d, the surfaces 720 in FIGS. 5a and 5b, the surfaces 820 in FIGS. 6a and 6b, the surface 920 in FIG. 7, the surfaces 320 in FIGS. 8b and 8c, the surfaces 420 in FIGS. 9b and 9c, the surface 1120 in FIG. 10, the surfaces 520 in FIGS. 11b and 11c, the surfaces 520a, 520b, 520c and 520d in FIG. 12, the surfaces 1220 in FIGS. 13a and 13c, the surfaces 1320a and 1320b in FIG. 14, surfaces 1420a, 1420b and 1420c in FIG. 15, and the surface 1720 in FIG. 17 do not have to be clear and they too can have undulating shape to provide mixing.

Throughout this application various publications have been referenced within parentheses. The disclosures of these publications in their entireties are hereby incorporated by reference in this application in order to more fully describe the state of the art to which this invention pertains.

It is understood that modifications which do not substantially affect the activity of the various embodiments of this invention are also included within the definition of the invention provided herein. Those skilled in the art will readily appreciate that the specific examples and studies detailed above are only illustrative of the invention. Accordingly, specific examples disclosed herein are intended to illustrate but not limit the present invention. It also should be understood that, although the invention has been described with reference to the disclosed embodiments, various modifications can be made without departing from the spirit of the invention. Accordingly, the invention is limited only by the following claims.

What is claimed is:

1. A mixing flow apparatus, comprising a waveguide and a mixing flow chamber;
said waveguide having a higher index of refraction material than its surroundings for propagation of a signal, and said mixing flow chamber having a body forming a flow chamber with an inlet, an outlet, a radiation transmissive wall and a surface shaped to disrupt flow regularity of a sample fluid, said body of said mixing flow chamber surrounding at least a portion of said waveguide, wherein constituents of a sample fluid entering said inlet are mixed by disruption of sample fluid flow regularity prior to discharge at said outlet.

2. A detection apparatus, comprising a waveguide, a mixing flow chamber and a radiation detector; said waveguide having a higher index of refraction material than its surroundings for propagation of a signal; said mixing flow chamber having a body forming a flow chamber with an inlet, an outlet, a radiation transmissive wall and a surface positioned to disrupt flow regularity of a sample fluid, said body of said mixing flow chamber surrounding at least a portion of said waveguide, wherein constituents of a sample fluid entering said inlet are mixed by disruption of sample fluid flow regularity prior to discharge at said outlet, and said radiation detector being disposed facing the direction of oncoming propagated signal from said waveguide.

3. The detection apparatus of claim 2, wherein a surface of said waveguide further comprises a reflective surface.

4. The detection apparatus of claim 2, wherein a surface of said waveguide further comprises an analyte recognition coating.

5. The detection apparatus of claim 4, further comprising one or more emission detection reagent species bound to said analyte recognition coating.

6. The detection apparatus of claim 4, wherein said analyte recognition coating further comprises an affinity binding reagent.

7. The detection apparatus of claim 4, wherein said analyte recognition coating further comprises a FRET detection reagent.

8. The detection apparatus of claim 4, wherein said surface of said waveguide comprises two or more different analyte recognition coatings.

9. The detection apparatus of claim 8, wherein said two or more analyte recognition coatings are spatially segregated on said surface.

10. The detection apparatus of claim 2, wherein said waveguide further comprises a material having a high index of refraction.

11. The detection apparatus of claim 2, wherein said waveguide or said mixing flow chamber further comprise a reflective element directing said signal towards said radiation detector.

12. The detection apparatus of claim 2, wherein said waveguide comprises a cross-sectional form selected from the group consisting of rectangular, circular, square, diamond, elliptical, polygonal, trapezoid, oval, a ring or any combination thereof.

13. The detection apparatus of claim 2, further comprising two or more waveguides.

14. The detection apparatus of claim 13, wherein said two or more waveguides are disposed parallel to each other.

15. The detection apparatus of claim 2, wherein said waveguide comprises said radiation transmissive surface.

16. The detection apparatus of claim 2, wherein said mixing flow chamber comprises an elongated flow chamber.

17. The detection apparatus of claim 16, wherein said inlet and outlet are distributed along a longitudinal axis of said elongated flow chamber sufficient to direct sample fluid flow along a length of the waveguide.

18. The detection apparatus of claim 16, wherein said inlets and outlets are distributed normal to a longitudinal axis of said elongated flow chamber sufficient to direct sample fluid flow perpendicular to the length of the waveguide.

19. The detection apparatus of claim 2, wherein said mixing flow chamber further comprises one or more flow guides, said flow guides facilitating directionality of sample fluid flow.

20. The detection apparatus of claim 19, wherein said flow guides direct sample fluid flow in a unidirectional longitudinal spiral.

21. The detection apparatus of claim 19, wherein said flow guides direct sample fluid flow in a unidirectional longitudinal zig-zag.

22. The detection apparatus of claim 2, wherein said disruption of sample fluid flow regularity comprises a non-laminar regime.

23. The detection apparatus of claim 2, wherein an internal surface of said mixing flow chamber comprises a radiation absorbing material.

24. The detection apparatus of claim 2, wherein an internal surface of said mixing flow chamber opposite of said radiation transmissive surface comprises a reflective surface.

25. The detection apparatus of claim 24, wherein said reflective surface is disposed parallel to a surface of said waveguide.

26. The detection apparatus of claim 2, wherein one or more portions of said mixing flow chamber comprise an undulating shape.

27. The detection apparatus of claim 26, further comprising two or more portions having an undulating shape.

28. The detection apparatus of claim 27, wherein said two or more undulating shapes are orientated in different direction.

29. The detection apparatus of claim 27, further comprising three or more differently orientated undulating shapes.

30. The detection apparatus of claim 2, wherein a longitudinal cross-section of said mixing flow chamber comprises a surface having a periodic shape.

31. The detection apparatus of claim 30, wherein said periodic shape of said body comprises a period of about 0.1 mm to about 15 mm.

32. The detection apparatus of claim 2, further comprising a separation of at least about 0.05 mm to about 5 mm between said portion of said waveguide and said body of said mixing flow chamber surrounding said portion of said waveguide.

33. The detection apparatus of claim 16, wherein said elongated shape further comprises an arc.

34. The detection apparatus of claim 2, further comprising one or more vessels capable of holding fluids.

35. The detection apparatus of claim 2, wherein said radiation detector comprises a spectrometer.

36. The detection apparatus of claim 2, further comprising a second radiation detector.

37. The detection apparatus of claim 36, wherein said second radiation detector is disposed facing a waveguide terminus different from a first radiation detector.

38. The detection apparatus of claim 2, further comprising an optical system disposed between said waveguide and said radiation detector.

39. The detection apparatus of claim 38, wherein said optical system comprises an optical filter system.

40. The detection apparatus of claim 2, wherein said radiation detector comprises detection of radiation power.

41. The detection apparatus of claim 40, wherein said detection of radiation power further comprises a function of radiation wavelength.

42. The detection apparatus of claim 2, wherein said radiation detector comprises detection of emitted radiation from one or more signals.

43. The detection apparatus of claim 40, wherein said detection of radiation power comprises instantaneous radiation power from a signal.

44. The detection apparatus of claim 40, wherein said detection of radiation power further comprises time integration.

45. The detection apparatus of claim 44, further comprising detection of a plurality of different signals.

46. The detection apparatus in claim 2, further comprising a fluid handling system capable of moving sample fluid through said mixing flow chamber.

47. The detection apparatus in claim 46, wherein said fluid handling system comprises an undulating structure or actuation device capable of moving sample fluid through said mixing flow chamber in forward and reverse directions.

48. The detection apparatus of claim 2, further comprising a plurality of waveguides, a plurality mixing flow chambers and a plurality radiation detectors; each of said plurality waveguides positioned within a mixing flow chamber of said plurality of mixing flow chambers; each of said mixing flow chambers having a body surrounding at least a portion of said waveguide positioned therein, wherein said body of each mixing flow chamber optically insulates waveguides positioned therein from scattered emission, uncaptured luminescence emission, or both, derived from waveguides positioned in one or more other mixing flow chambers within said plurality of mixing flow chambers.

49. A signal detection apparatus, comprising a waveguide, a mixing flow chamber, a radiation detector and an illumination source;
said waveguide having a higher index of refraction material than its surroundings for propagation of a signal;
said mixing flow chamber having a body forming a flow chamber with an inlet, an outlet, a radiation transmissive wall and a surface positioned to disrupt flow regularity of a sample fluid, said body of said mixing flow chamber surrounding at least a portion of said waveguide member, wherein constituents of a sample fluid entering said inlet are mixed by disruption of sample fluid flow regularity prior to discharge at said outlet;
said radiation detector being disposed facing the direction of outgoing propagated signal from said waveguide, and
said illumination source being positioned to direct electromagnetic radiation on one or more surfaces of said waveguide.

50. The detection apparatus of claim 49, wherein a surface of said waveguide further comprises a reflective surface.

51. The detection apparatus of claim 49, wherein a surface of said waveguide further comprises an analyte recognition coating.

52. The detection apparatus of claim 51, further comprising one or more fluorescent species bound to said analyte recognition coating.

53. The detection apparatus of claim 51, further comprising one or more phosphorescence species bound to said analyte recognition coating.

54. The detection apparatus of claim 51, wherein said analyte recognition coating further comprises an affinity binding reagent.

55. The detection apparatus of claim 51, wherein said further comprises a FRET detection reagent.

56. The detection apparatus of claim 51, wherein said surface of said waveguide comprises two or more different analyte recognition coatings.

57. The detection apparatus of claim 56, wherein said two or more analyte recognition coatings are spatially segregated on said surface.

58. The detection apparatus of claim 49, wherein a surface of said waveguide further comprises a material having a high index of refraction.

59. The detection apparatus of claim 49, wherein said waveguide or said mixing flow chamber further comprise a reflective element directing said signal towards said radiation detector.

60. The detection apparatus of claim 49, wherein said waveguide comprises a cross-sectional form selected from the group consisting of rectangular, circular, square, diamond, elliptical, polygonal, trapezoid, oval, and a ring.

61. The detection apparatus of claim 49, further comprising two or more waveguides.

62. The detection apparatus of claim 61, wherein said two or more waveguides are disposed parallel to each other.

63. The detection apparatus of claim 49, wherein said waveguide comprises said radiation transmissive surface.

64. The detection apparatus of claim 49, wherein said mixing flow chamber comprises an elongated flow chamber.

65. The detection apparatus of claim 64, wherein said inlet and outlet are distributed along a longitudinal axis of said elongated flow chamber sufficient to direct sample fluid flow along a length of the waveguide.

66. The detection apparatus of claim 64, wherein said inlets and outlets are distributed normal to a longitudinal axis of said elongated flow chamber sufficient to direct sample fluid flow perpendicular to the length of the waveguide.

67. The detection apparatus of claim 49, wherein said mixing flow chamber further comprises one or more flow guides, said flow guides facilitating directionality of sample fluid flow.

68. The detection apparatus of claim 67 wherein said flow guides direct sample fluid flow in a unidirectional longitudinal spiral.

69. The detection apparatus of claim 67, wherein said flow guides direct sample fluid flow in a unidirectional longitudinal zig-zag.

70. The detection apparatus of claim 49, wherein said disruption of sample fluid flow regularity comprises a non-laminar regime.

71. The detection apparatus of claim 49, wherein an internal surface of said mixing flow chamber comprises a radiation absorbing material.

72. The detection apparatus of claim 49, wherein an internal surface of said mixing flow chamber opposite of said radiation transmissive surface comprises a reflective surface.

73. The detection apparatus of claim 72, wherein said reflective surface is disposed parallel to a surface of said waveguide.

74. The detection apparatus of claim 49, wherein one or more portions of said mixing flow chamber comprise an undulating shape.

75. The detection apparatus of claim 74, further comprising two or more portions having an undulating shape.

76. The detection apparatus of claim 75, wherein said two or more undulating shapes are orientated in different direction.

77. The detection apparatus of claim 75, further comprising three or more differently orientated undulating shapes.

78. The detection apparatus of claim 49, wherein a longitudinal cross-section of said mixing flow chamber comprises a surface having a periodic shape.

79. The detection apparatus of claim 78, wherein said periodic shape of said body comprises a period of about 0.1 mm to about 15 mm.

80. The detection apparatus of claim 49, further comprising a separation of at least about 0.1 mm to about 5 mm between said portion of said waveguide and said body of said mixing flow chamber surrounding said portion of said waveguide.

81. The detection apparatus of claim 64, wherein said elongated shape further comprises an arc.

82. The detection apparatus of claim 49, further comprising one or more vessels capable of holding fluids.

83. The detection apparatus of claim 49, wherein said radiation detector comprises a spectrometer.

84. The detection apparatus of claim 49, further comprising a second radiation detector.

85. The detection apparatus of claim 84, wherein said second radiation detector is disposed facing a waveguide terminus different from a first radiation detector.

86. The detection apparatus of claim 49, further comprising an optical system disposed between said waveguide and said radiation detector.

87. The detection apparatus of claim 86, wherein said optical system comprises an optical filter system.

88. The detection apparatus of claim 49, wherein said radiation detector comprises detection of radiation power.

89. The detection apparatus of claim 88, wherein said detection of radiation power further comprises a function of radiation wavelength.

90. The detection apparatus of claim 49, wherein said radiation detector comprises detection of emitted radiation from one or more signals.

91. The detection apparatus of claim 88, wherein said detection of radiation power comprises instantaneous radiation power from a signal.

92. The detection apparatus of claim 88, wherein said detection of radiation power further comprises time integration.

93. The detection apparatus of claim 92, further comprising detection of a plurality of different signals.

94. The detection apparatus in claim 49, further comprising a fluid handling system capable of moving sample fluid through said mixing flow chamber.

95. The detection apparatus in claim 94, wherein said fluid handling system comprises an undulating structure or actuation device capable of moving sample fluid through said mixing flow chamber in forward and reverse directions.

96. The detection apparatus of claim 49, further comprising a plurality of waveguides, a plurality mixing flow chambers and a plurality radiation detectors; each of said plurality waveguides positioned within a mixing flow chamber of said plurality of mixing flow chambers; each of said mixing flow chambers having a body surrounding at least a portion of said waveguide positioned therein, wherein said body of each mixing flow chamber optically insulates waveguides positioned therein from scattered emission, uncaptured luminescence emission, or both, derived from waveguides positioned in one or more other mixing flow chambers within said plurality of mixing flow chambers.

97. The signal detection apparatus of claim 49, wherein said illumination source comprises a projection beam impinging substantially perpendicular to a surface of said waveguide.

98. The signal detection apparatus of claim 97, wherein radiation from said projection beam is substantially collimated.

99. The signal detection apparatus of claim 49, further comprising two or more illumination sources.

100. The signal detection apparatus of claim 99, wherein said two or more illumination sources further comprise a projection beam of radiation, each of said projection beams impinging on an analyte recognition coating on said waveguide.

101. The signal detection apparatus of claim 100, further comprising each of said projection beams impinging on two or more different analyte recognition coatings.

102. The signal detection apparatus of claim 101, wherein said two or more different analyte recognition coatings are spatially segregated on said waveguide.

103. The signal detection apparatus of claim 49, further comprising an optical system disposed between the waveguide and said illumination source.

104. The signal detection apparatus of claim 103, wherein said optical system comprises a configuration uniformly irradiating a surface of a waveguide.

105. The signal detection apparatus of claim 103, wherein said optical system further comprises an optical filter system.

106. The signal detection apparatus of claim 105, wherein said optical filter system comprises a wavelength selection filter.

107. An analyte sensing apparatus, comprising:
a radiation illumination member;
a radiation detector member; and
a mixing flow cartridge member, comprising a waveguide member having a first end and a second end and multiple surfaces, wherein at least a first surface of the waveguide is facing the analyte fluid sample; and
a mixing flow chamber member having a body comprising at least a radiation transmissive side, a first end and a second end, an inlet and an outlet connected to said body, the body of said mixing flow chamber surrounding at least part of said waveguide member and spaced therefrom so as to allow a fluid solution to flow between the inlet and the outlet, wherein the first end and the second end of waveguide member are secured to first and second end of the mixing flow chamber body;
wherein the first surface of the waveguide member is coated with an analyte recognition element, wherein the waveguide and the body of the mixing flow chamber is configured so that the sample fluid flow through the mixing flow chamber produces mixing of the sample, wherein the radiation illumination member constructed to provide electromagnetic radiation and arranged to direct the radiation on the analyte recognition elements on the surfaces of the waveguide member, and wherein one radiation detector member is disposed facing the first end of the waveguide.

108. A method for detecting an analyte in a fluid sample, comprising:
(a) flowing a sample fluid suspected of containing an analyte over an analyte recognition coating of a signal detection apparatus under conditions sufficient for binding of an analyte to said analyte recognition coating, said signal detection apparatus comprising:
a waveguide having an analyte recognition coating, said waveguide having a higher index of refraction material than its surroundings for propagation of a signal;
a mixing flow chamber having a body forming a flow chamber with an inlet, an outlet, a radiation transmissive wall and a surface positioned to disrupt flow regularity of a sample fluid, said body of said mixing flow chamber surrounding at least a portion of said waveguide member, wherein constituents of a sample fluid entering said inlet are mixed by disruption of sample fluid flow regularity prior to discharge at said outlet;
a radiation detector being disposed facing the direction of outgoing propagated signal from said waveguide, and
an illumination source being positioned to direct electromagnetic radiation on one or more surfaces of said waveguide.
(b) flowing an emission detection reagent over said analyte recognition coating under conditions sufficient for binding of said detection reagent to a bound analyte;
(c) exposing said analyte recognition coating to radiation having a wavelength sufficient to excite said detection reagent, and
(d) detecting emitted radiation with said radiation detector.

109. The method of claim 108, further comprising the step:
(a1) removing unbound analyte.

110. The method of claim 108, wherein said optical emission detection reagent comprises a luminescent, fluorescent or phosphorescent detection reagent.

111. The method of claim 110, wherein said detection reagent further comprises a FRET detection reagent.

112. A method for detecting an analyte in a fluid sample, comprising:
(a) contacting a sample fluid suspected of containing an analyte with an analyte recognition coating in solution under conditions sufficient for binding of an analyte to said analyte recognition coating;
(b) immobilizing said analyte recognition coating to a waveguide of a signal detection apparatus, said signal detection apparatus comprising:
a waveguide having an analyte recognition coating, said waveguide having a higher index of refraction material than its surroundings for propagation of a signal;
a mixing flow chamber having a body forming a flow chamber with an inlet, an outlet, a radiation transmissive wall and a surface positioned to disrupt flow regularity of a sample fluid, said body of said mixing flow chamber surrounding at least a portion of said waveguide member, wherein constituents of a sample fluid entering said inlet are mixed by disruption of sample fluid flow regularity prior to discharge at said outlet;
a radiation detector being disposed facing the direction of outgoing propagated signal from said waveguide, and
an illumination source being positioned to direct electromagnetic radiation on one or more surfaces of said waveguide.
(c) contacting said analyte recognition coating with an emission detection reagent under conditions sufficient for binding of said detection reagent to a bound analyte;
(d) exposing said analyte recognition coating to radiation having a wavelength sufficient to excite said detection reagent, and
(e) detecting emitted radiation with said radiation detector.

113. The method of claim 112, wherein said contacting with an emission detection reagent in step (c) occurs simultaneously with step (a).

114. The method of claim 112, wherein said contacting with an emission detection reagent in step (c) occurs during or after said immobilization in step (b).

115. The method of claim 112, further comprising the step after (a):
(a1) removing analyte recognition coating or emission detection reagent.

116. The method of claim 112, wherein said emission detection reagent comprises a luminescent, fluorescent or phosphorescent detection reagent.

117. The method of claim 116, wherein said emission detection reagent further comprises a FRET detection reagent.
